SEQUENCE LISTING (2 sequences)

SEQ ID NO: 1 - DNA, 30 bp, Artificial Sequence
  Description of Artificial Sequence PCR primer for amplifying CD44 gene
tttaagcttatggacaagttttggtggcac

SEQ ID NO: 2 - DNA, 32 bp, Artificial Sequence
  Description of Artificial Sequence PCR primer for amplifying CD44 gene
ttttctagaaacacgtcatcatcagtagggtt

(19) United States
(12) Patent Application Publication
(10) Pub. No.: US 2006/0211651 A1
(43) Pub. Date: Sep. 21, 2006

(54) Persulfated oligosaccharide acting on selectins and chemokine

(75) Inventors: Masayuki Miyasaka, Takatsuki-shi (JP); Hiroto Kawashima, Kawaguchi-shi (JP)

Correspondence Address: BIRCH STEWART KOLASCH & BIRCH, PO BOX 747, FALLS CHURCH, VA, 22040-0747, US

(73) Assignee: Taisho Pharmaceutical Co., Ltd., Tokyo (JP)

(21) Appl. No.: 10/522,549

(22) PCT Filed: Jan. 29, 2003
(86) PCT No.: PCT/JP03/00823

§ 371 Date: Jan. 27, 2005

(30) Foreign Application Priority Data
Jul. 29, 2002 (JP) 2002-220301

(51) Int. Cl. A61K 31/737; C08B 37/00

(52) U.S. Cl. 514/54; 536/53

(57) ABSTRACT

A persulfated saccharide compound capable of acting on selectin and chemokine. According to the present invention, there are provided a saccharide compound capable of interacting with L-selectin, P-selectin and chemokine; a pharmaceutical composition; and an agent for treating or preventing a disease of which sideration is associated with biological events mediated by any of L-selectin, P-selectin and chemokine.

### TECHNICAL FIELD

The present invention relates to a persulfated oligosaccharide capable of acting on selectins and chemokines. Particularly, the present invention relates to a saccharide compound which interacts with L-selectin, P-selectin and chemokine which are an pro-inflammatory molecule, as well as a therapeutic or prophylactic agent for a disease of which sideration is associated with biological events mediated by any of L-selectin, P-selectin and chemokine. More particularly, the present invention relates to an agent for treating or preventing a disease of which sideration is associated with biological events mediated with L-selectin, P-selectin, chemokine or the like; a lead compound for drug discovery for the therapeutic or prophylactic agent; a saccharide compound useful for designing or the like of the lead compound, as well as a therapeutic or prophylactic agent useful for treating or preventing a disease such as inflammatory disease, allergic disease, cancer metastasis, myocardial dysfunction and multiple organ failure.

### BACKGROUND ART

It is known that inflammation is induced by an adhesion molecule on leukocytes and a molecule for promoting adhesion with a vascular endothelial cell, and that certain adhesion molecules are involved in inflammation. The adhesion molecule includes, for instance, a selectin family which is represented by L-selectin and P-selectin.

It is known that, in the interaction between the L-selectin or P-selectin and its ligand, sulfation of the ligand plays an important role. For example, it is known that tyrosine sulfation of P-selectin glycoprotein ligand-1 is necessary for the interaction between the P-selectin glycoprotein ligand-1 and any of L-selectin and P-selectin [Sako, D. et al., Cell, 83, 323-331 (1995); Pouyani, T. et al., Cell, 83, 333-343 (1995); and Spertini, O. et al., J. Cell Biol., 135, 523-531 (1996)].

In addition, it is known that a ligand of L-selectin on a high endothelial small vein binds to L-selectin in a sulfation-dependent manner [Imai, Y. et al., Nature, 361, 555-557 (1993); Hiraoka, N. et al., Immunity, 11, 79-89 (1999); Bistrup, A. et al., J. Cell Biol., 145, 899-910 (1999)]. Further, it is known that HNK-1 reactive sulfoglucronyl glycolipid [Needham, L. K. et al., Proc. Natl. Acad. Sci. U.S.A., 90, 1359-1363 (1993)], heparin oligosaccharide [Nelson, R. M. et al., Blood, 11, 3253-3258 (1993)] and heparan sulfate glycosaminoglycan [Koenig, A. et al., J. Clin. Invest., 101, 877-889 (1998)] binds to any of L-selectin and P-selectin.

However, since any of compounds which binds to any of L-selectin and P-selectin have a high molecular weight, under the circumstances, it is difficult to design a lead compound capable of regulating a binding between L-selectin, P-selectin or the like and a ligand and that a medicament using the compound has not yet been successfully developed.

### DISCLOSURE OF INVENTION

An object of the present invention is to provide a saccharide compound, by which the regulation of binding between any of L-selectin, P-selectin and chemokine and its ligand, the regulation of biological events mediated by any of L-selectin, P-selectin and chemokine, improvement in symptom of a disease of which sideration is associated with the biological events, and provision of a lead compound for a therapeutic or a prophylactic agent for the disease can be achieved. Also, an object of the present invention is to provide an agent for treating or preventing a disease of which sideration is associated with biological events mediated by any of L-selectin, P-selectin and chemokine, which can improve symptom of a disease such as inflammatory disease, allergic disease, cancer metastasis, myocardial disorder and multiple organ failure, and can exhibit high affinity in a living body.

Specifically, the present invention relates to
[1] a saccharide compound represented by the general formula (I): 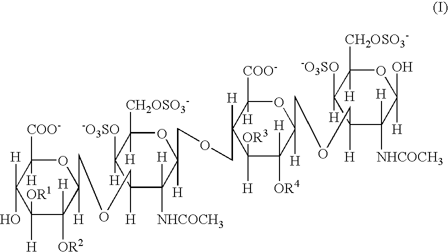
wherein R¹, R², R³ and R⁴ each independently represent a hydrogen atom or a sulfonic group, or the general formula (II): 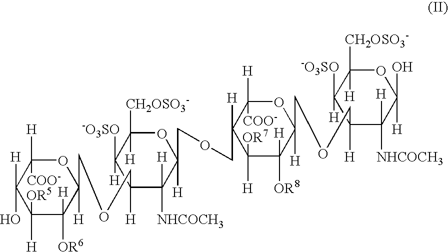
wherein R⁵, R⁶, R⁷ and R⁸ each independently represent a hydrogen atom or a sulfonic group;
[2] a saccharide compound represented by the general formula (III): 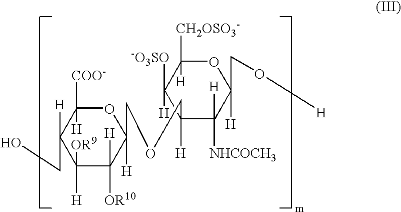
wherein R⁹ and R¹⁰ each independently represent a hydrogen atom or a sulfonic group, and m is 3 or 4, or the general formula (IV): 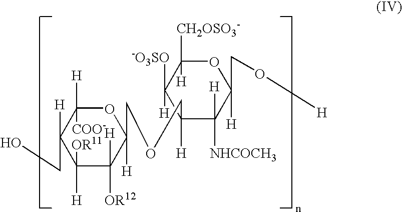
wherein R¹¹ and R¹² each independently represent a hydrogen atom or a sulfonic group, and n is 3 or 4;
[3] a pharmaceutical composition comprising the saccharide compound of the above item [1] or [2] as an active ingredient; and
[4] an agent for treating or preventing a disease of which sideration is associated with biological events mediated by any of L-selectin, P-selectin and chemokine, wherein said agent comprises the saccharide compound of the above item [1] or [2] as an active ingredient.

### BRIEF DESCRIPTION OF THE DRAWINGS

FIG. 1 is a view showing the results of investigation by immunoprecipitation for the effects of sulfation on the interaction between a chondroitin sulfate/dermatan sulfate chain in versican and each of L-selectin, P-selectin and CD44.

FIG. 2 is a view showing the results of investigation by enzyme-linked immunosorbent assay for the effects of a persulfated CS/DS chain on binding between versican and any of L-selectin, P-selectin and CD44. In FIG. 2, a cross shows keratan sulfate, an open triangle being chondroitin, a open square being chondroitin sulfate A, a open circle being chondroitin polysulfate, a solid triangle being dermatan, a solid square being dermatan sulfate, a solid circle-solid line being dermatan polysulfate, and a solid circle-dashed line being chondroitin sulfate E. In addition, in FIG. 2, panel A is the results for binding between versican and L-selectin, panel B being the results for binding between versican and P-selectin, and panel C being the results for binding between versican and CD44.

FIG. 3 is a view showing a disaccharide composition of versican-derived glycosaminoglycan. Panel A shows the results of analysis of a disaccharide composition of versican-derived glycosaminoglycan was treated with chondroitinase ABC and then derivatized with 2-aminobenzamide (2-AB). Panel B shows the results of analysis of a disaccharide composition of versican-derived glycosaminoglycan which was treated with chondro-6-sulfatase and chondroitinase ABC and then derivatized with 2-AB. In addition, in FIG. 3, 0S shows the elution position of 2-AB-derivatized ΔDi-0S, 4S being the elution position of 2-AB-derivatized ΔDi-4S, 6S being the elution position of 2-AB-derivatized ΔDi-6S, diS_{D} being the elution position of 2-AB-derivatized ΔDi-di(2,6)S, diS_{E} being the elution position of 2-AB-derivatized ΔDi-di(4,6)S, and UA2S being the elution position of 2-AB-derivatized ΔDi-UA2S.

FIG. 4 is a view showing the results of investigation for the effects of sulfation on the interaction between versican and chemokine. Panel A shows the disaccharide composition of a product derivatized with 2-aminobenzamide. 0S shows the elution position of 2-AB-derivatized ΔDi-0S, 4S being the elution position of 2-AB-derivatized ΔDi-4S, 6S being the elution position of 2-AB-derivatized ΔDi-6S, diS_{D} being an elution position of 2-AB-derivatized ΔDi-di(2,6)S, and diS_{E} being an elution position of 2-AB-derivatized ΔDi-di(4,6)S. Panel B shows the results of investigation by enzyme-linked immunosorbent assay for the effects of sulfation on the interaction between versican and chemokine. BSA represents bovine serum albumin, 2B1 being anti-versican monoclonal antibody 2B1, L-Ig being L-selectin-Ig, E-Ig being E-selectin-Ig, P-Ig being P-selectin-Ig, SLC being secondary lymphoid tissue chemokine, SLC-T being C-terminal truncated secondary lymphoid tissue chemokine, IP-10 being γ-interferon inducible protein-10, PF4 being platelet factor 4, SDF-1β being stromal cell-derived factor-1β, and SDF-1α being stromal cell-derived factor-1α. Each of the bars in panel B shows mean±standard deviation from tetraplicate measurement. A black bar is the results for untreated conditioned medium-derived versican, and a hatched bar being the results for sodium chlorate-treated conditioned medium-derived versican. Each of the bars shows mean±standard deviation from tetraplicate measurement.

FIG. 5 is a view showing the results of investigation by enzyme-linked immunosorbent assay for the effects of a persulfated CS/DS chain on binding between versican and chemokine. Expression of an abscissa denotes the same as that of FIG. 4. In addition, in each of lanes, bar 1 shows the results in the absence of glycosaminoglycan, bar 2 being chondroitin, bar 3 being chondroitin sulfate A, bar 3 being chondroitin polysulfate, bar 5 being dermatan polysulfate, and bar 6 being chondroitin sulfate E. Each of the bars shows mean±standard deviation from triplicate measurement.

FIG. 6 shows the sensorgram of BIAcore, in which the interaction between immobilized glycosaminoglycan and each of chemokine, L-selectin and CD44 is recorded. In FIG. 6, SLC represents secondary lymphoid tissue chemokine, IP-10 being γ-interferon inducible protein-10, SDF-1β being stromal cell-derived factor-1β, CS E being chondroitin sulfate E, and CS A being chondroitin sulfate A. In FIG. 6, the response in a resonance unit is recorded as a function of time.

FIG. 7 is a view showing the results of identification of the structure of a fragment which interacts with each of L-selectin, P-selectin and chemokine. Panel A shows HPLC chromatogram of a hyaluronidase-digested product of each of chondroitin sulfate A (CS A in the figure), chondroitin sulfate C(CS C in the figure) and chondroitin sulfate E (CS E in the figure). Panel B shows a disaccharide composition of each of the fraction a, the fraction c, the fraction e-1 and the fraction e-2. Panel C is a schematic view of the structure of each of the fraction a, the fraction c, the fraction e-1 and the fraction e-2, a solid triangle shows GlcA, a hatched circle being GalNAc, 4S being 4-O-sulfation, 6S being 6-O-sulfation, β3 being β1-3 linkage, and β4 being β1-4 linkage. Panel D shows results of investigation for the interaction between an oligosaccharide contained in each of the fraction a, the fraction c, the fraction e-1 and the fraction e-2, and each of L-selectin, P-selectin, CD44 and chemokine. The expression of an abscissa denotes the same as that of FIG. 4. In addition, in each of lanes, bar 1 shows the results of the case where streptoavidin-conjugated alkaline phosphatase is used, bar 2 being the results of the case where streptoavidin-conjugated alkaline phosphatase coupled to biotinylated fraction a is used, bar 3 being the results of the case where streptoavidin-conjugated alkaline phosphatase coupled to biotinylated fraction c is used, bar 4 being the results of the case where streptoavidin-conjugated alkaline phosphatase coupled to biotinylated fraction e-1 is used, and bar 5 being the results of the case where streptoavidin-conjugated alkaline phosphatase coupled to biotinylated fraction e-2 is used. Each of the bars denotes mean±standard deviation from tetraplicate measurement.

FIG. 8 is a view showing the results of investigation for the effects of a persulfated CS/DS chain on chemokine activity. In FIG. 8, SLC represents secondary lymphoid tissue chemokine, SLC-T being C-terminal truncated secondary lymphoid tissue chemokine, CS E being chondroitin sulfate E, and CS A being chondroitin sulfate A. In addition, in FIG. 8, the arrowhead shows the time point at which stimulation was given.

### BEST MODE FOR CARRYING OUT THE INVENTION

The present invention is based on surprising findings that a saccharide compound comprising, as a constituent unit, a disaccharide moiety of the general formula (III′): 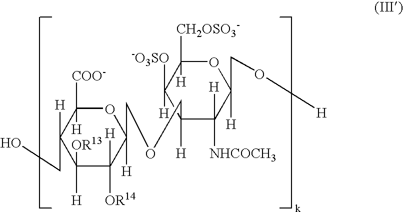
wherein R¹³ and R¹⁴ each independently represent a hydrogen atom or a sulfonic group, and k is an arbitrary natural number, or
a disaccharide moiety of the general formula (IV′): 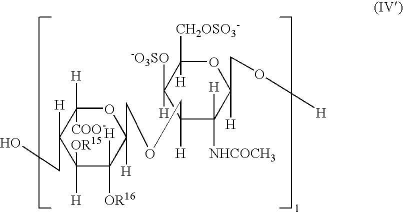
wherein R¹⁵ and R¹⁶ each independently represent independently a hydrogen atom or a sulfonic group, and l is an arbitrary natural number,
among sulfated glycosaminoglycans, in particular, a tetrasaccharide compound having a repeat unit of GlcAβ1-3GalNAc(4,6-O-disulfate) or a tetrasaccharide compound having a repeat unit of IdoAα1-3GalNAc(4,6-O-disulfate) interacts with each of L-selectin, P-selectin and chemokine, and surprising findings that the tetrasaccharide compounds inhibit physiological activities of chemokine.

In the specification, “GlcA” represents D-glucronate residue, “GalNAc” being N-acetyl-D-glactosamine residue, “IdoA” being L-iduronate residue, and “HexA” being hexuronate residue. In addition, “β1-3” means β1-3 linkage, and “α1-3” means α1-3 linkage.

The saccharide compound of the present invention includes a saccharide compound represented by the general formula (I): 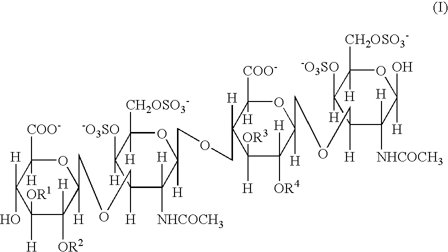
wherein R¹, R², R³ and R⁴ each independently represent a hydrogen atom or a sulfonic group, or

a saccharide compound represented by the general formula (II): 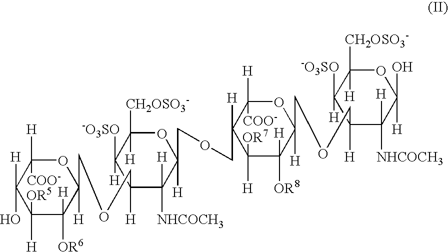
wherein R⁵, R⁶, R⁷ and R⁸ each independently represent a hydrogen atom or a sulfonic group, and

a saccharide compound represented by the general formula (III): 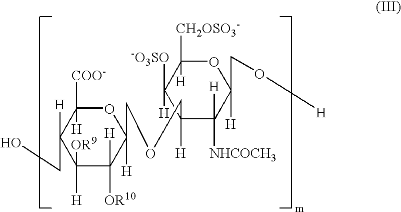
wherein R⁹ and R¹⁰ each independently represent a hydrogen atom or a sulfonic group, and m is 3 or 4, or

a saccharide compound represented by the general formula (IV): 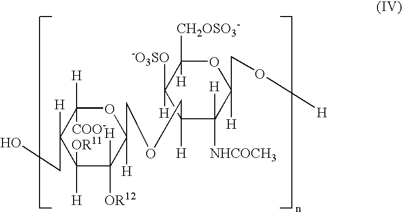
wherein R¹¹ and R¹² each independently represent a hydrogen atom or a sulfonic group, and n is 3 or 4. Although the saccharide compound of the present invention is a compound comprising one of a tetra- to octasaccharide, there are exhibited excellent effects that the saccharide compound interacts with any of L-selectin, P-selectin and chemokine, to regulate physiological functions associated with any of L-selectin, P-selectin and chemokine. In addition, the saccharide compound of the present invention can be simply prepared upon preparation thereof. Further, since the saccharide compound of the present invention is a compound comprising one of a tetra- to octasaccharide, there are exhibited excellent effects that it can be used as a low molecular compound capable of regulating binding between at least one kind selected from the group consisting of L-selectin, P-selectin and chemokine, and the ligand thereof, or as a basis for designing lead compound thereof. In addition, according to the saccharide compound of the present invention, there can be carried out the regulation of binding between any of L-selectin, P-selectin and chemokine, and the ligand thereof, the regulation of biological events mediated by any of L-selectin, P-selectin and chemokine, the improvement of symptom of a disease of which sideration is associated with the biological events, and the provision of a lead compound for a remedy or an inventive for the disease.

In the present invention, from the viewpoint of a molecular weight, particularly, a saccharide compound represented by the above general formula (I) or general formula (II) is preferable.

In the above general formula (I), R¹, R², R³ and R⁴ each independently represent a hydrogen atom or a sulfonic group. In addition, the sulfonic group may have a substituent, as long as the object of the present invention is not hindered. A saccharide compound in which any of R¹, R², R³ and R⁴ in the general formula (I) is a sulfonic group can be obtained, for example, with a sulfur trioxide-trialkylamine complex, a sulfur trioxide-pyridine complex, a sulfuric acid-trialkylamine complex or a sulfuric acid-pyridine complex.

In addition, in the general formula (II), R⁵, R⁶, R⁷ and R⁸ each independently represent a hydrogen atom or a sulfonic group. The sulfonic group may have a substituent, as long as the object of the present invention is not hindered. A compound in which any of R⁵, R⁶, R⁷ and R⁸ in the general formula (II) is a sulfonic group can be obtained, for example, with a sulfur trioxide-trialkylamine complex, a sulfur trioxide-pyridine complex, a sulfuric acid-trialkylamine complex or a sulfuric acid-pyridine complex.

Further, in the above general formula (III), R⁹ and R¹⁰ each independently represent a hydrogen atom or a sulfonic group. The sulfonic group may have a substituent, as long as the object of the present invention is not hindered. A compound in which R⁹ and/or R¹⁰ in the above general formula (III) is a sulfonic group can be obtained, for example, with a sulfur trioxide-trialkylamine complex, a sulfur trioxide-pyridine complex, a sulfuric acid-trialkylamine complex or a sulfuric acid-pyridine complex.

In addition, in the general formula (IV), R¹¹ and R¹² each independently represent a hydrogen atom or a sulfonic group. The sulfonic group may have a substituent, as long as the object of the present invention is not hindered. A compound in which R¹¹ and/or R¹² is (are) a sulfonic group in the general formula (IV) can be obtained, for example, by a sulfur trioxide-trialkylamine complex, a sulfur trioxide-pyridine complex, a sulfuric acid-trialkylamine complex or a sulfuric acid-pyridine complex.

Concretely, the saccharide compound of the present invention includes, GlcAβ1-3GalNAc(4,6-O-disulfate)β1-4GlcAβ1-3GalNAc(4,6-O-sulfate), IdoAα1-3GlNAc(4,6-O-disulfate)β1-4IdoAα1-3GalNAc(4,6-O-disulfate), GlcAβ1-3GalNAc(4,6-O-disulfate)β1-4GlcAβ1-3GalNAc(4,6-O-sulfate)β1-4GlcAβ1-3 GalNAc(4,6-O-sulfate), IdoAα1-3GlNAc(4,6-O-disulfate)β1-4IdoAα1-3GalNAc(4,6-O-disulfate)β1-4IdoAα1-3GalNAc(4,6-O-disulfate), GlcAβ1-3GalNAc(4,6-O-disulfate)β1-4GlcAβ1-3GalNAc(4,6-O-sulfate)β1-4GlcAβ1-3 GalNAc(4,6-O-sulfate)β1-4GlcAβ1-3GalNAc(4,6-O-sulfate), IdoAα1-3GlNAc(4,6-O-disulfate)β1-4IdoAα1-3GalNAc(4,6-O-disulfate)β1-4IdoAα1-3GalNAc(4,6-O-disulfate)β1-4IdoAα1-3GalNAc(4,6-O-disulfate), and the like. Among the above-mentioned saccharide compounds, a compound having a high degree of sulfation is desirable from the viewpoint of sufficient exhibition of the interactions with L-selectin, P-selectin and chemokine.

The saccharide compound of the present invention can be obtained by performing the steps of:

(a) digesting a persulfated chondroitin sulfate/dermatan sulfate chain found in a squid cartilage [Suzuki, S. et al., J. Biol. Chem., 243, 1543-1550 (1968)], a mast cell [Katz, H. R. et al., J. Biol. Chem., 261, 13393-13396 (1986); and Stevens, R. L. et al., Proc. Natl. Acad. Sci. U.S.A., 85, 2284-2287 (1988)], neutrophil [Ohhashi, Y. et al., Biochem. J., 217, 199-207 (1984); and Petersen. F. et al., J. Biol. Chem., 274, 12376-12382 (1999)], monocyte [Uhlin-Hansen, L. et al., J. Biol. Chem., 264, 14916-14922 (1989); McGee, M. P. et al., J. Biol. Chem., 270, 26109-26115 (1995)], glomerulus [Kobayashi, S. et al., Biochim. Biophy. Acta, 841, 71-80 (1985)], and glomerulus stromal cell [Yaoita, E. et al., J. Biol. Chem., 265, 522-531 (1990)], with, for example, hyaluronidase, and

(b) subjecting the digestion product obtained in the step (a) to high performance liquid chromatography, to thereby obtain an oligosaccharide fraction.

In the step (a), a persulfated chondroitin sulfate/dermatan sulfate chain can be obtained by a conventional procedure. In addition, a source of the persulfated chondroitin sulfate/dermatan sulfate chain may be, but not particularly limited to the above-exemples, other organisms, tissues, and cells, which carry a persulfated chondroitin sulfate/dermatan sulfate chain.

Then, in the step (b), the digestion product obtained in the above (a) is subjected to chromatography, for example, high performance liquid chromatography. In the step (b), various conventional chromatographies may be performed so as to obtain a single kind of oligosaccharide as a single peak. For example, in the case of squid cartilage-derived chondroitin A, squid cartilage-derived chondroitin C and squid cartilage-derived chondroitin E, a combination of amine-coupled silica gel column chromatography using a linear gradient of 16 mM to 1M NaH₂PO₄, and subsequent gel filtration column chromatography can be performed as the above-described chromatography. Here, a solution used in chromatography may be an aqueous solution or a mixed solution of a water-soluble organic solvent and water, which is suitable for separating the oligosaccharide and the like.

The saccharide compound obtained in the above step (b) may be sulfated with a sulfur trioxide-trialkylamine complex, a sulfur trioxide-pyridine complex, a sulfuric acid-trialkylamine complex or a sulfuric acid-pyridine complex. In particular, it is desirable to perform 6-O-sulfation of chondroitin sulfate, or 6-O-sulfation of dermatan sulfate, preferably, by the method of Nagasawa [Nagasawa, K. et al., Carbohydr. Res., 158, 183-190 (1986); all teachings of which are hereby incorporated by reference].

The saccharide compound of the present invention exhibits the properties:

\textcircled{1} interacting, for example, binding with any of L-selectin, P-selectin and chemokine;

\textcircled{2} regulating, specifically, inhibiting the binding between versican and any of L-selectin, P-selectin and chemokine;

\textcircled{3} inhibiting physiological activities of chemokine.

For this reason, it is expected that the saccharide compound of the present invention is applied to treatment or prevention of a disease of which sideration is associated with biological events mediated with any of L-selectin, P-selectin and chemokine.

Therefore, according to the present invention, there is provided a pharmaceutical composition comprising the saccharide compound in the present invention as an active ingredient. The pharmaceutical composition may further comprise a carrier (pharmaceutically acceptable carrier) capable of maintaining stably the effective ingredient, a pharmaceutically acceptable auxiliary agent, excipient, binder, stabilizer, buffer, solubilizer, and isotonic agent, depending on the disease to be applied, the status of the disease, and the individual, organ, local site and a tissue to be administered.

A content of the effective ingredient in the pharmaceutical composition of the present invention can be appropriately set depending on a disease to be applied, status of the disease and an individual, an organ, a local site and a tissue to be administered and can be set, for example, in the same manner as that of the therapeutic or prophylactic agent described later.

Further, the evaluation of the pharmaceutical composition of the present invention can be performed in the same manner as that of the therapeutic or prophylactic agent described later.

Further, according to the present invention, there is provided an agent for treating or preventing a disease of which sideration is associated with biological events mediated by any of L-selectin, P-selectin and chemokine.

One of the features of the therapeutic or prophylactic agent of the present invention resides in that the agent comprises the saccharide compound of the present invention as an active ingredient.

The “biological events mediated by any of L-selectin, P-selectin and chemokine” includes, for example, the infiltration in tissue in inflammation, the regulation of cytokine production, lymphocyte homing, platelet aggregation, vascularization of tumor lesion, cancer metastasis, myocardial ischemia reperfusion disease and the like. In addition, the above disease includes inflammatory disease, infectious disease, asthma, allergic inflammation, stromal pneumonia, systemic inflammatory response syndrome, and inflammatory disease and the like.

According to the therapeutic or prophylactic agent of the present invention, since the agent comprises the saccharide compound of the present invention as an active ingredient, there are exhibited excellent effects such that the therapeutic or prophylactic agent interacts with each of L-selectin, P-selectin and chemokine, to regulate, specifically, to inhibit eliciting inflammation in inflammatory disease, for example, adhesion of leukocyte with vascular endothelial cell mediated by the L-selectin and/or P-selectin, and to regulate, specifically, to inhibit physiological activities of chemokine (the regulation of cytokine production, Ca²⁺ mobilization and the like). In addition, according to the therapeutic or prophylactic agent of the present invention, since the therapeutic or prophylactic agent comprises the saccharide compound of the present invention as an active ingredient, there can be regulated the binding between at least one kind selected from the group consisting of L-selectin, P-selectin and chemokine, and the ligand thereof. Therefore, according to the therapeutic or prophylactic agent, there are exhibited excellent effects such that there can be carried out treatment of inflammatory disease, allergic disease, and cancer metastasis associated with biological events mediated by any of L-selectin, P-selectin and chemokine, or inhibition or prevention of the symptom thereof. Namely, the therapeutic or prophylactic agent of the present invention can be used as an anti-inflammatory agent, an antiallergic agent, an anti-cancer agent and the like. Further, according to the therapeutic or prophylactic agent of the present invention, there are exhibited excellent properties such that the agent improves symptom of a disease such as inflammatory disease, allergic disease, cancer metastasis, myocardial dysfunction, and multiple organ failure, and shows high affinity in a living body. Therefore, there is exhibited an excellent effect such that the agent can improve symptom of a disease of which sideration is associated with biological events mediated by any of L-selectin, P-selectin and chemokine.

Evaluation of the therapeutic or prophylactic agent of the present invention can be performed, for example, as follows:

Action of the therapeutic or prophylactic agent of the present invention, for example, an ability to inhibit the binding, can be evaluated, for example, by surface plasmon resonance analysis described below. Namely, the action of the therapeutic or prophylactic agent of the present invention can be evaluated by performing an evaluation method comprising the steps of:

(i) immobilizing L-selectin, P-selectin or chemokine on a sensor chip, to obtain each of a L-selectin-immobilized sensor chip, a P-selectin-immobilized sensor chip and a chemokine-immobilized sensor chip,

(ii) loading a ligand for each of L-selectin, P-selectin and chemokine on any of a L-selectin-immobilized sensor chip, a P-selectin-immobilized sensor chip and a chemokine-immobilized sensor chip, corresponding to the ligand, at a constant flow rate in the presence of the therapeutic or prophylactic agent of the present invention, and

(iii) detecting the interaction as an optical variation or a mass variation by an appropriate detecting means [e.g. optical detection (fluorescence, fluorescent polarization degree and the like), in combination with mass spectrometer (matrix-assisted laser desorption ionization time of flight mass spectrometer: MALDI-TOF MS, electrospray-ionization mass spectrometer: ESI-MS), thereby obtaining a sensorgram.

In the step (i), a ligand may be immobilized on a sensor chip and, in this case, in the step (ii), any of L-selectin, P-selectin and chemokine may be loaded on a corresponding ligand-immobilized sensor chip at a constant flow rate in the presence of the therapeutic or prophylactic agent.

In the evaluation method, in the case where a sensorgram indicating the formation of a complex of each of L-selectin, P-selectin and chemokine with the ligand thereof in the absence of the therapeutic or prophylactic agent of the present invention is shown, for example, the case where an optical sensorgram or a mass sensorgram is varied by introduction of a ligand by liquid pumping, serves as a negative control. Therefore, inhibition of binding by the therapeutic or prophylactic agent of the present invention is recognized as an index, the case where a sensorgram indicating the formation of a complex is not shown or the case where a time until the formation of a complex is delayed, in the presence of the therapeutic or prophylactic agent of the present invention in the same reaction system as in the case of the absence of the therapeutic or prophylactic agent of the present invention.

The action of the therapeutic or prophylactic agent of the present invention, for example, the regulation of biological events mediated by any of L-selectin, P-selectin, and chemokine can be evaluated by determining the presence or the absence, or an extent of biological events in a cultured cell in the presence of the therapeutic or prophylactic agent of the present invention. For example, regarding Ca²⁺ mobilization mediated with secondary lymphoid tissue chemokine, the action can be evaluated by performing an evaluation method comprising the steps of:

i) applying Fura-2 to stimulate to L1.2/CCR7 cell (1×10⁶ cells/ml) with secondary lymphoid tissue chemokine in the presence or absence of the therapeutic or prophylactic agent (100 μg/ml) of the present invention, and

ii) measuring a fluorescent rate to monitor an intracellular calcium concentration according to the description of Hirose, J. et al. [J. Biol. Chem., 276, 5228-5234 (2001); all teachings of which are hereby incorporated by reference].

Further, the action of the therapeutic or prophylactic agent of the present invention, for example, a therapeutic or preventive effects on an inflammatory disease in a living body can be evaluated by administering the therapeutic or prophylactic agent of the present invention to an inflammatory disease model animal, and then observing a change in symptom at an inflammatory site; by detecting neutrophil infiltration using activity of tissue myeloperoxidase as an index.

A dosage form of the therapeutic or prophylactic agent of the present invention can be appropriately selected depending on the administration form. The dosage form includes, for example, a medicine for oral administration such as a tablet; a medicine for external application such as a spray medicine and an ointment; injection for subcutaneous, intradermal intramuscular, or intravenous injection, and the like.

Therefore, the content of an effective ingredient in the therapeutic or prophylactic agent of the present invention can be appropriately set depending on, for example, an age, a weight, pathology and the like of an individual in need of treatment or prevention of the above disease. It is desired that, for example, the content is 10 to 500 mg, when the administration form is intravenous injection and that the content is 10 to 500 mg, when the administration form is subcutaneous injection.

In addition, a dose of the therapeutic or prophylactic agent of the present invention to an individual can be appropriately set depending on, for example, the age, weight, pathology and the like of an individual in need of treatment or prevention of the above disease. It is desired that, for example, the amount of an effective ingredient is 1 μg/kg (weight of individual) to 10 mg/kg (weight of individual), preferably 100 μg/kg (weight of individual) to 10 mg/kg (weight of individual), more preferably 1 mg/kg (weight of individual) to 8 mg/kg (weight of individual).

The therapeutic or prophylactic agent of the present invention may further comprise a pharmaceutically acceptable auxiliary agent, excepient, binder, stabilizer, buffer, solubilizer and isotonic, depending on status of a disease of which sideration is associated with biological events mediated by any of L-selectin, P-selectin and chemokine, and the individual, organ, local site and tissue to be administered and the like.

In addition, the therapeutic or prophylactic agent of the present invention may be an agent obtained by making a carrier (pharmaceutically acceptable carrier) capable of stably maintaining the effective ingredient to carry the effective ingredient. Specifically, for example, the effective ingredient may be carried by a pharmaceutically acceptable carrier which can facilitate introduction into a living body such as an individual, organ, local site and tissue to be administered.

Further, according to the present invention, there is provided a method for treating or preventing a disease associated with biological events mediated by any of L-selectin, P-selectin and chemokine, specifically, for example, a method for treating inflammatory disease, allergic disease, cancer metastasis, myocardial dysfunction, multiple organ failure and the like, inhibiting or preventing symptom thereof. The method for treatment or prevention of the present invention can be performed according to a dosage form and a dose of the above therapeutic or prophylactic agent.

The present invention will be explained in more detail below by Examples, but the present invention is not limited by the Examples. Unless otherwise is indicated, reagents used in the present Examples are the same as those described in Kawashima, H. et al. [J. Biol. Chem., 275, 35448-35456 (2000)] and Hirose, J. et al. [J. Biol. Chem., 276, 5228-5234 (2001)]. In addition, for a composition of a cell culture medium etc., see Frshney, R. Ian, Culture of animal cell: A manual of basic technique, 2nd ed., Alan R. Liss. Inc., 66-84 (1987), all teachings of which are hereby incorporated by reference.

### EXAMPLE 1

Requirement of sulfation in the interaction between a chondroitin sulfate/dermatan sulfate chain (hereinafter, also referred to as CS/DS chain) in versican, and any of L-selectin, P-selectin and CD44 was examined by treating ACHN cells with a metabolism inhibitor for sulfation, sodium chlorate.

(1) Preparation of Human CD44-Ig

Human-derived CD44 cDNA was prepared by PCR using a sense primer (SEQ ID NO: 1):

| | |
|---|---|
| 5′-TTTAAGCTTATGGACAAGTTTTGGTGGCAC-3′ | (SEQ ID NO: 1) |

wherein the bold face is HindIII restriction enzyme recognition site, and the underlined portion is codons for initial 7 amino acids of human CD44,

and an antisense primer (SEQ ID No.: 2):

(SEQ ID NO: 2)

5′-TTTTCTAGAAACACGTCATCATCAGTAGGGTT-3′

wherein the bold face is XbaI restriction enzyme recognition site, and the underlined portion is codons of amino acids positions 172 to 178 of human CD44.
The resulting amplification product was inserted into a cloning site of the expression vector pcDNA 3.1/Myc-His(+) B [manufactured by Invitrogen], to obtain a human CD44 expression vector. In addition, the amplification product was sequenced.

293T cells were transfected with the human CD44 expression vector, using the LipofectAMINE™ reagent [manufactured by Invitrogen] according to the instruction by the manufacturer. The resulting transfected cells were cultured at 37° C. for 4 days in the Dulbecco's modified Eagle medium containing 20% by weight of bovine fetal serum in a CO₂ incubator, to obtain a conditioned medium.

Thereafter, the conditioned medium was subjected to immunoaffinity chromatography with anti-CD44 monoclonal antibody BRIC 235-coupled CNBr-activated Sepharose 4B, to obtain monomeric CD44. The resulting soluble CD44 was subjected to SDS-PAGE and silver staining. As a result, the purity of the resulting soluble CD44 was more than 95%.

Then, using the resulting CD44, human CD44-immunoglobulin (Ig) was prepared according to the method described in Toyama-Sorimachi, N. [J. Biol. Chem., 270, 7437-7444, (1995); all teachings of which are hereby incorporated by reference].

(2) Labeling of Metabolism of ACHN Cells Using [³⁵S]Sodium Sulfate or [³⁵S]Methionine.

ACHN cells were pre-incubated at 37° C. for 6 hours in RPMI 1640 containing 10% by weight of bovine fetal serum in the presence or absence of 30 mM sodium chlorate. A confluent monolayer of the resulting ACHN cells was labeled by incubation for 18 hours in the presence or absence of 30 mM sodium chlorate, in a medium of either of:

(i) Eagle minimum basal medium SMEM [sulfate-free; manufactured by BioWhittaker] containing 2% by weight of dialyzed bovine fetal serum in the presence of 0.2 mCi/ml [³⁵S]Na₂SO₄ [trade name: Sulfer-35; manufactured by ICN Radiochemicals], or

(ii) methionine-free RPMI 1640 medium [manufactured by Invitrogen] containing 2% by weight of dialyzed bovine fetal serum in the presence of 0.2 mCi/ml [³⁵S]-methionine [manufactured by ICN Radiochemicals].

(3) Immunoprecipitation

Immunoprecipitation was performed as follows, according to the method described in the publication of Kawashima, H. et al. [Int. Immunol., 11, 393-405 (1999); all teachings of which are hereby incorporated by reference] except that beads were washed using buffer A (0.05% Tween 20, 20 mM HEPES-NaOH, 0.15M NaCl, 1 mM CaCl₂, 1 mM MgCl₂, pH6.8).

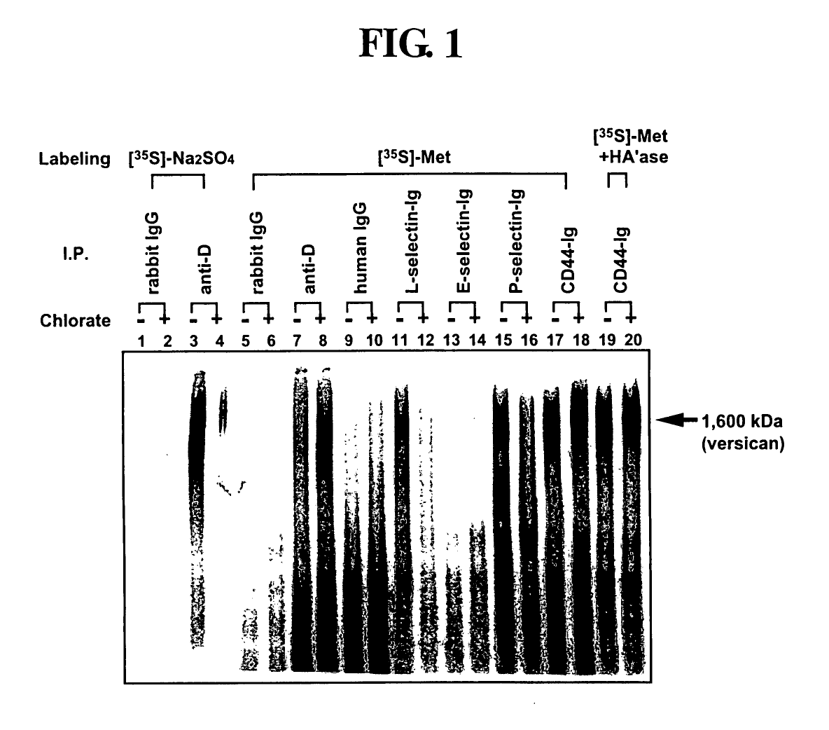

Each of the antibody and the Ig chimera shown in FIG. 1 (equivalent to 10 μg) was coupled to Protein A-Sepharose column (equivalent to 10 μL) by a conventional method, to obtain antibody-coupled Protein A-Sepharose or Ig chimera-coupled Protein A-Sepharose. Then, each antibody-coupled Protein A-Sepharose or each Ig chimera-coupled Protein A-Sepharose (equivalent to 10 μl gel), and the conditioned medium were incubated at 4° C. overnight in 1 ml of the buffer A. Anti-D [Zimmermann, D. R. et al., J. Cell. Biol., 124, 817-825 (1994)] which is an anti-versican polyclonal antibody was provided by Dr. Dietel R. Zimmermann (Institute of Clinical Pathology, University of Zuerich), and human L-selectin-Ig was manufactured by R & D Systems Inc.

Thereafter, the beads were washed with the buffer A, to obtain precipitates. The resulting precipitates were subjected to SDS-agarose-PAGE, and thereafter, signal ascribed to ³⁵S label on a gel was detected by autoradiography. The results are shown in FIG. 1.

As shown in lanes 5 to 8 of FIG. 1, although a core protein of versican was synthesized, as shown in lanes 1 to 4, treatment with sodium chlorate resulted in more than 90% inhibition of sulfation relative to the case without treatment.

In addition, as shown in lanes 11, 12, 15 and 16 of FIG. 1, the above sodium chlorate treatment inhibited the interaction between versican and each of L-selectin-Ig and P-selectin-Ig. Therefore, it was suggested that sulfation of CS/DS chain of versican is necessary for the interaction between versican and each of L-selectin and P-selectin. On the other hand, as shown in lanes 13 and 14 of FIG. 1, unsulfated versican did not interact with E-selectin-Ig, which is consistent with the results in Kawashima, H. et al. [J. Biol. Chem., 275, 35448-35456, (2000)].

In contrast, as shown in lanes 17 and 18 of FIG. 1, the interaction between versican and CD44-Ig was not inhibited by sodium chlorate treatment. However, the fact that the interaction between versican and CD44-Ig was not inhibited by sodium chlorate treatment was contemplated to be due to the formation of a trimolecular complex composed of versican, hyaluronic acid and CD44-Ig.

Then, [³⁵S]methionine-labeled conditioned medium of ACHN cells was treated with hyaluronidase SD (in FIG. 1, “HA′ase”; 50 mU/ml) for 3 hours in the same manner as described in the publication [Kawashima, H. et al., J. Biol. Chem., 275, 35448-35456, (2000); all teachings of which are hereby incorporated by reference], and then the resulting conditioned medium and CD44-Ig were incubated.

As the result, although the hyaluronic acid was almost completely degraded and removed by the above treatment with hyaluronidase SD, but as shown in lanes 19 and 20 of FIG. 1, the interaction between versican and CD44-Ig was not affected at all. Therefore, it was confirmed that sulfation of versican is not necessary for the interaction of versican and CD44, namely, a CS/DS chain of versican interacts with CD44 in a sulfation independent manner.

### EXAMPLE 2

The structure for interacting with each of selectin and CD44 was examined by performing Inhibition assay using various CS/DS chains.

(1) Analysis of Disaccharide Composition of CS/DS Chain

A CS/DS chain used in the inhibition assay was chondroitin (“CH” in Table 1), chondroitin sulfate A (“CS A” in Table 1), chondroitin polysulfate (“CPS” in Table 1), dermatan, dermatan sulfate purified from a cock's comb [“DS” in Table 1; manufactured by SEIKAGAKU CORPORATION; Nagasawa, K. et. al., Carbohydr. Res., 131, 301-314, (1984)], dermatan polysulfate (“DPS” in Table 1), and chondroitin sulfate E (“CS E” in Table 1). The above chondroitin was produced by chemical desulfurization of chondroitin sulfate A derived from whale cartilage [Nagasawa, K., J. Biochem., 86, 1323-1329 (1979)]. The above chondroitin polysulfate (manufactured by SEIKAGAKU CORPORATION) was produced by selective 6-O-sulfation of chondroitin sulfate A [Nagasawa, K. et al., Carbohydr. Res., 158, 183-190 (1986)]. The above dermatan polysulfate (manufactured by SEIKAGAKU CORPORATION) was produced by selective 6-O-sulfation of dermatan sulfate [Nagasawa, K. et al., Carbohydr. Res., 158, 183-190 (1986)]. Dermatan (manufactured by SEIKAGAKU CORPORATION) was produced by the above chemical desulfurization of dermatan sulfate [Nagasawa, K., J. Biochem., 86, 1323-1329 (1979)].

The CS/DS chain was treated with chondroitinase ABC (1 unit/ml) at 37° C. for 7 hours in the same manner as that of Fujimoto, T. et al. [Int. Immunol., 13, 359-366 (2001); all teachings of which are hereby incorporated by reference]. The resulting product was subjected to HPLC with amine-coupled silica PA-03 column, to thereby determine the disaccharide composition. Results are shown in Table 1.

TABLE 1

| | ΔDi- | ΔDi- | ΔDi- | ΔDi-di | ΔDi-di | ΔDi-di | ΔDi-tri |
|---|---|---|---|---|---|---|---|
| | 0S | 6S | 4S | (2,6)S | (2,4)S | (4,6)S | (2,4,6)S |
| CH | 94.7 | 2 | 3.3 | | | | |
| CS A | 2.2 | 23.7 | 74.1 | | | | |
| CPS | | 26.9 | 3.3 | 10.9 | | 47.3 | 11.5 |
| Dermatan | 85.8 | | 14.2 | | | | |
| DS | 5 | 3.4 | 85.2 | | 6.3 | | |
| DPS | 1.8 | 9.1 | 1.5 | 2.9 | | 62.1 | 21.2 |
| CS E | 5.8 | 9.7 | 18.7 | | | 65.9 | |

Although a chain length and an atomic valence of chondroitin polysulfate, those of chondroitin sulfate A and those of chondroitin are identical, degrees of sulfation of these glycosaminoglycans are different. Similarly, dermatan polysulfate, dermatan sulfate and dermatan are different only in sulfation.

(2) Binding Inhibition Assay

Inhibition assay of binding between versican and each of L-selectin, P-selectin and CD44 was performed as follows. First, each well on a 96 well flat bottom microtiter plate (Coster EIA/RIA plate No. 3690) was coated with L-selectin-Ig (3 μg/ml), P-selectin-Ig (4 μg/ml) or CD44-Ig (0.25 μg/ml) by a conventional procedure. Then, various concentrations of each of keratan sulfate, chondroitin, chondroitin sulfate A, chondroitin polysulfate, dermatan, dermatan sulfate, dermatan polysulfate and chondroitin sulfate E, and biotinylated versican was added to the wells on the plate. The plate was incubated at room temperature for 2 hours. Thereafter, the plate was washed with the buffer A, and enzyme-linked immunosorbent assay was performed in the same manner as described in Kawashima, H. et al. [J. Biol. Chem., 275, 35448-35456 (2000)]. Binding was determined by measuring absorbance at 620 nm using alkaline phosphatase-conjugated streptavidin and Blue Phos™ substrate.

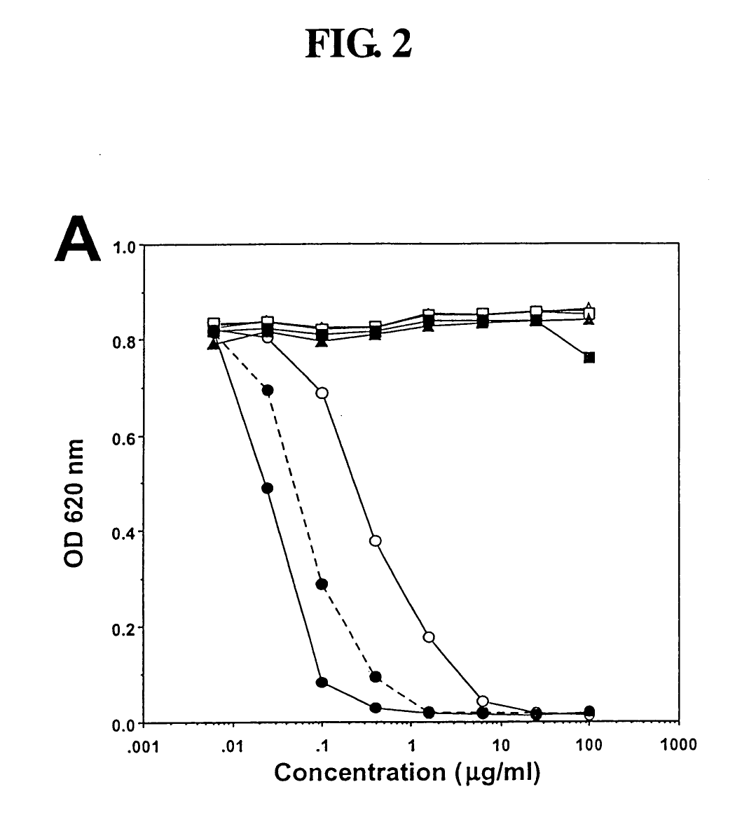

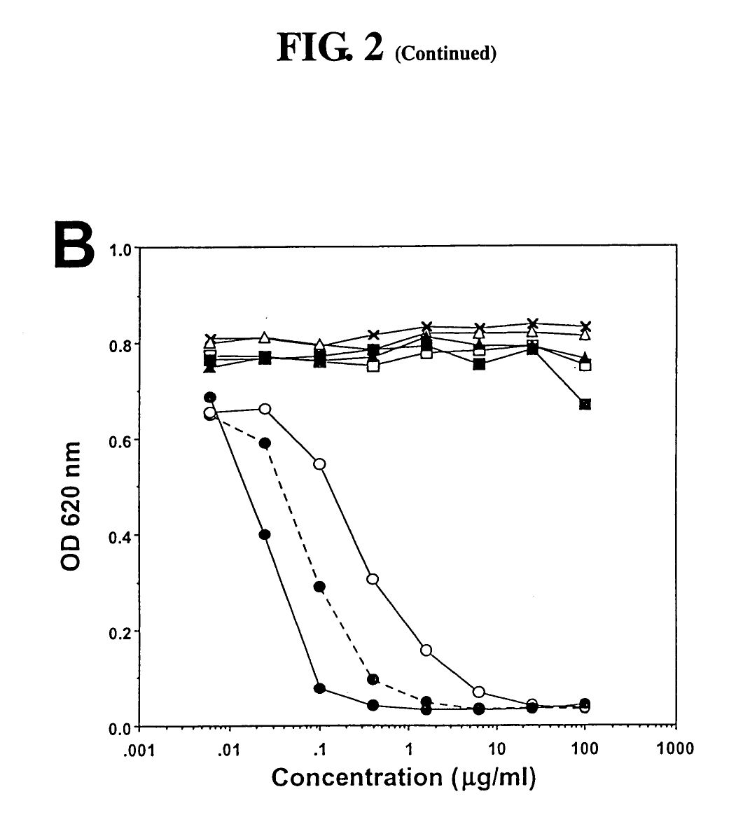

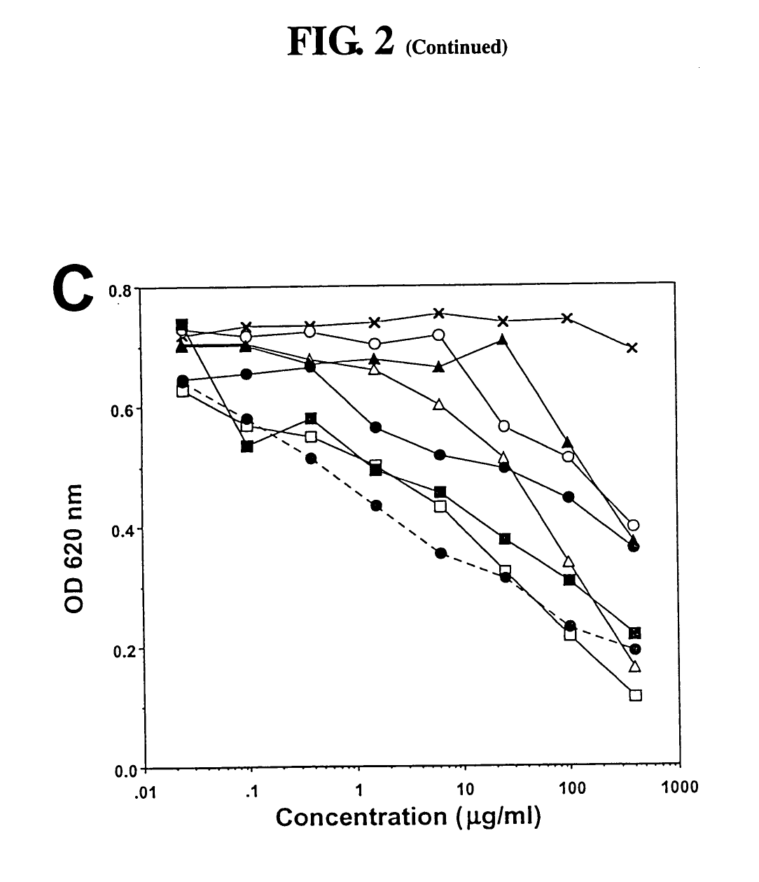

Results are shown in FIG. 2. In FIG. 2, a cross denotes keratan sulfate, an open triangle denotes chondroitin, an open square denotes chondroitin sulfate A, an open circle denotes chondroitin polysulfate, a solid triangle denotes dermatan, a solid square denotes dermatan sulfate, a solid circle-solid line denotes dermatan polysulfate, and a solid circle-dashed line denotes chondroitin sulfate E. In FIG. 2, panel A is the results for binding between versican and L-selectin, panel B being the results regarding binding between versican and P-selectin, and panel C being the results regarding binding between versican and CD44.

As shown in FIG. 2, binding between biotinylated versican and each of L-selectin-Ig and P-selectin-Ig is inhibited by glycosaminoglycan containing GlcAβ1/IdoAα1-3GalNAc(4,6-O-disulfate) as a main disaccharide component (chondroitin polysulfate, dermatan polysulfate and chondroitin sulfate E) in a dose-dependent manner, but was not inhibited by a low-sulfated or unsulfated CS/DS chain such as chondroitin, chondroitin sulfate A, dermatan, and dermatan sulfate, or keratan sulfate. In contrast, binding between biotinylated versican and CD44-Ig was inhibited by all of CS/DS chains examined such as low-sulfated CS/DS chain, and unsulfated CS/DS chain, but was not inhibited by keratan sulfate.

These results were consistent with the results of the above Example 1 that sulfation plays an important role in the interaction between CS/DS chain of versican and each of L-selectin and P-selectin, but does not play a role in the interaction between a CD/DS chain of versican and CD44.

### EXAMPLE 3

The glycosaminoglycan structure of versican was characterized.

(1) Preparation of Versican-Derived Glycosaminoglycan

Purified versican (80 μg) was incubated at 37° C. for 48 hours in 2 ml of a solution (composition: 5 mM Tris-HCl, 5 mM CH₃COONa, 1 mM CaCl₂, 1 mM MgCl₂, pH8.0) containing pronase [90 U or more, manufactured by Calbiochem]. After incubation, 0.5 ml of a solution (composition: 1M NaBH₄, 5M NaOH) was added to the resulting product, followed by incubation at 37° C. After incubation for 24 hours, 0.5 ml of CH₃COOH was added to the resulting product to terminate the reaction, to thereby obtain the reaction mixture containing versican-derived glycosaminoglycan. The resulting reaction mixture was dialyzed against distilled water using a Spectra/Por dialysis membrane [molecular weight exclusion limit 3,500; manufactured by The Spectrum Co.], to obtain versican-derived glycosaminoglycan.

(2) High Sensitive Disaccharide Composition Analysis of Versican-Derived Glycosaminoglycan

The versican-derived glycosaminoglycan obtained in the above item (1) and chondroitinase ABC (0.38 U/ml) were incubated at 37° C. for 1 hour in the presence or the absence of chondro-6-sulfatase [0.3]U/ml buffer B (3% acetic acid adjusted to pH 7.0 with triethylamine)], and thereafter, the resulting product was dried to obtain a disaccharide product. Then, the disaccharide product (0.1 to 50 nmol) and 5 μl of a derivatization reagent mixture [composition: 0.35M 2-aminobenzoamide (2-AB), 0.1M NaCNBH₄, 30% by weight acetic acid in dimethyl sulfoxide] were mixed and then incubated at 65° C. for 2 hours according to the method of Kinoshita et al. [Kinoshita A. et al., Anal. Biochem., 269, 367-378 (1999)]. Then, the resulting product was fractionated with chloroform:distilled water (1:1), to collect the aqueous layer containing a derivatized disaccharide.

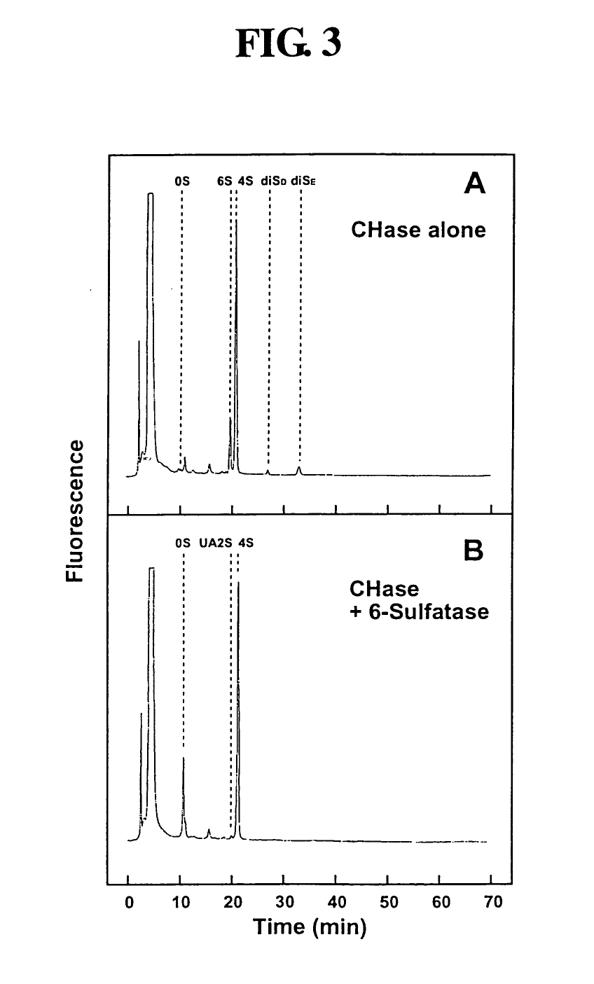

The resulting aqueous layer containing a derivatized disaccharide was analyzed by high performance liquid chromatography (HPLC) as described in that of the previous report [Fujimoto, T. et al., Int. Imminol., 13, 359-366 (2001)]. Regarding the extract, each of the excision wavelength at 330 nm and the emission wavelength at 420 nm was monitored using a fluorescent detector. The results are shown in FIG. 3. In FIG. 3, 0S denotes an elution position of 2-AB-derivatized ΔDi-0S, 4S denotes an elution position of 2-AB-derivatized ΔDi-4S, 6S denotes an elution position of 2-AB-derivatized ΔDi-6S, diS_{D} denotes an elution position of 2-AB-derivatized ΔDi-di(2,6)S, diS_{E} denotes an elution position of 2-AB-derivatized ΔDi-di(4,6)S, and UA2S denotes an elution position of 2-AB-derivatized ΔDi-UA2S. In addition, a table of abbreviations of disaccharide specimens is shown in Table 2.

TABLE 2

| Abbreviation | Sugar Chain |
|---|---|
| ΔDi-0S | Δ^{4,5}HexAα1-3GalNAc |
| ΔDi-4S | Δ^{4,5}HexAα1-3GalNAc(4-O-sulfate) |
| ΔDi-6S | Δ^{4,5}HexAα1-3GalNAc(6-O-sulfate) |
| ΔDi-di(2,6)S | Δ^{4,5}HexA(2-O-sulfate)α1-3GalNAc(6-O-sulfate) |
| ΔDi-di(2,4)S | Δ^{4,5}HexA(2-O-sulfate)α1-3GalNAc(4-sulfate) |
| ΔDi-di(4,6)S | Δ^{4,5}HexAα1-3GalNAc(4,6-O-disulfate) |
| ΔDi-tri(2,4,6)S | Δ^{4,5}HexA(2-O-sulfate)α1-3GalNAc(4,6-O-disulfate) |
| ΔDi-UA2S | Δ^{4,5}HexA(2-O-sulfate)α1-3GalNAc |
| Di-0S | GlcAβ1-3 GalNAc |
| Di-4S | GlcAβ1-3GalNAc(4-O-sulfate) |
| Di-6S | GlcAβ1-3GalNAc(6-O-sulfate) |
| Di-di(4,6)S | GlcAβ1-3GalNAc(4,6-O-disulfate) |

As shown in panel A in FIG. 3, five peaks ascribed to elution positions of disaccharide specimens were detected for versican-derived glycosaminoglycan treated with chondroitinase ABC. In addition, as shown in panel B of FIG. 3, three peaks ascribed to ΔDi-0S, ΔDi-UA-2S and ΔDi-4S were detected for versican-derived glycosaminoglycan treated with chondro-6-sulfatase and chondroitinase ABC. Therefore, it was confirmed that the five peaks detected in panel A of FIG. 3 are ΔDi-0S, ΔDi-6S, ΔDi-4S, ΔDi-di(2,6)S and ΔDi-di(4,6)S.

The peak area of each ΔDi-0S, ΔDi-6S, ΔDi-4S, ΔDi-di(2,6)S and ΔDi-di(4,6)S in panel A of FIG. 3 was 0.8%, 15.7%, 77.6%, 1.4% and 4.5%. Although a small additional peak (6.3%) not ascribed to a disaccharide specimen was detected, the similar results was also obtained using chondroitinase ACII in place of chondroitinase ABC. From these results, it was suggested that glycosaminoglycan of versican contains GlcAβ1-3GalNAc(4,6-O-disulfate), and is a heteropolymer composed of a mixture of a major CS chain, and a minor DS chain which is resistant to chondroitinase ACII.

### EXAMPLE 4

It is shown that versican can interact not only with an adhesion molecule but also with a certain chemokine [Hirose, J. et al., J. Biol. Chem., 276, 5228-5234 (2001)]. Then, the requirement of sulfation for the interaction between versican and chemokine was examined.

(1) Preparation of Low-Sulfated Versican

ACHN cells were cultured for 2 days in the presence or absence of 100 mM sodium chlorate in RPMI 1640 containing 10% by weight of bovine fetal serum. After the conditioned medium was removed, cells were further cultured for 4 days in a serum-free medium, EX-CELL 610 HSF [manufactured by JRH Bioscience] in the presence or absence of 100 mM sodium chlorate. The conditioned medium was recovered, and spun at 10,000×g at 4° C. for 15 minutes to obtain each of a sodium chlorate-treated conditioned medium and sodium chlorate-untreated conditioned medium.

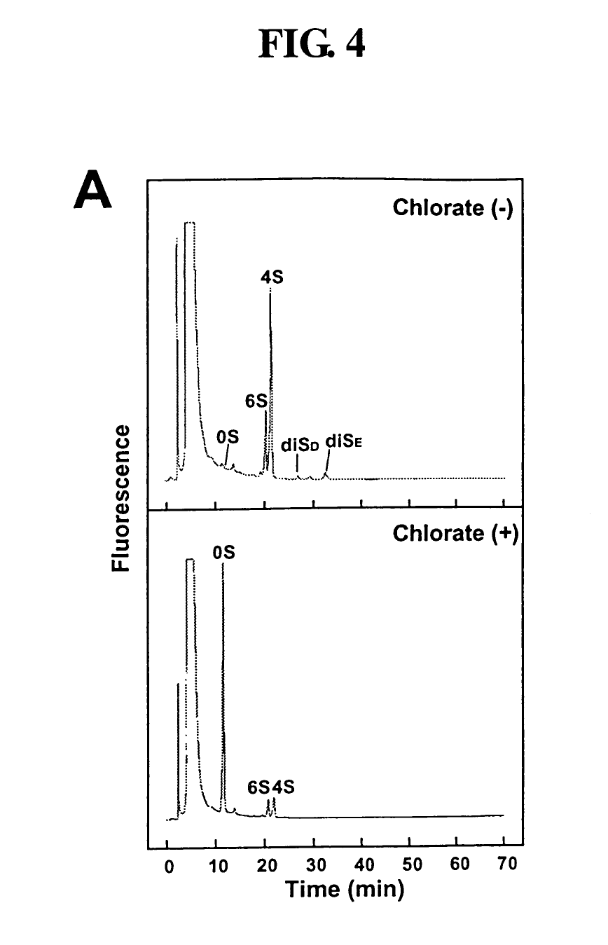

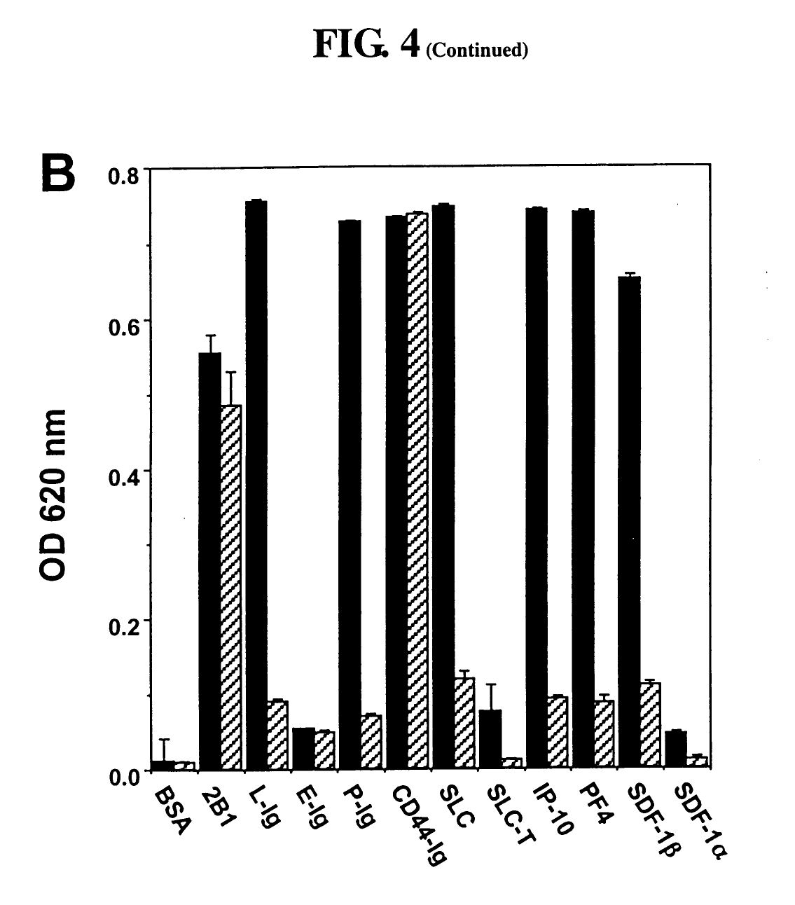

The sodium chlorate-treated conditioned medium or the sodium chlorate untreated conditioned medium, and 20 turbidity reduction unit/ml hyaluronidase (Streptomyces hyalurolyticus) were incubated at 37° C. for 4.5 hours. Thereafter, each of the resulting products, and anti-D antibody (10 μg)-coupled Protein A-Sepharose beads (10 μl beads) was incubated, to precipitate versican from each conditioned medium. The resulting beads were washed, and incubated at 37° C. for 2 hours in the presence of 1 unit/ml chondroitinase ABC and 1 unit/ml chondroitinase ACII. Thereafter, the disaccharide product was recovered, and derivatized with 2-AB according to the above method of Kinoshita et al. The resulting derivatized disaccharide product was analyzed by HPLC. The results are shown in panel A of FIG. 4. In panel A of FIG. 4, 0S denotes an elution position of 2-AB-derivatized ΔDi-0S, 4S being an elution position of 2-AB-derivatized ΔDi-4S, 6S being an elution position of 2-AB-derivatized ΔDi-6S, diS_{D} being an elution position of 2-AB-derivatized ΔDi-di(2,6)S, and diS_{E} being an elution position of 2-AB-derivatized ΔDi-di(4,6)S.

As shown in panel A of FIG. 4, in the case of versican-derived glycosaminoglycan obtained from the sodium chlorate-treated conditioned medium, only ΔDi-0S was detected as a main peak (82.8%) in versican-derived glycosaminoglycan, ΔDi-6S (7.2%) and ΔDi-4S (10.0%) were detected as a minor peak, and ΔDi-di(2,6)S or ΔDi-di(4,6) was not detected. Therefore, it is found that sodium chlorate treatment provides mainly low-sulfated versican capable of generating a unsulfated CS/DS chain.

(2) Sandwich-Type Enzyme-Linked Immunosorbent Assay

Binding between each of the resulting low-sulfated versican and intact versican, and chemokine was examined by sandwich-type enzyme-linked immunosorbent assay.

A well of a 96 well flat bottom microtiter plate was coated with BSA (6 μg/ml), anti-versican monoclonal antibody 2B1 (“2B1” in FIG. 4; 5 μg/ml), L-selectin-Ig (“L-Ig” in FIG. 4; 3 μg/ml), E-selectin-Ig (“E-Ig” in FIG. 4; 3 μg/ml), P-selectin-Ig (“P-Ig” in FIG. 4; 3 μg/ml), CD44-Ig (3 μg/ml), secondary lymphoid tissue chemokine (“SLC” in FIG. 4; 3 μg/ml), C-terminal truncated secondary lymphoid tissue chemokine (“SLC-T” in FIG. 4; 3 μg/ml), γ-interferon inducible protein-10 (“IP-10” in FIG. 4; 3 μg/ml), platelet factor 4 (“PF4” in FIG. 4; 6 μg/ml), stromal cell-derived factor-1β (“SDF-1β” in FIG. 4; 6 μg/ml) or stromal cell-derived factor-1α (“SDF-1α” in FIG. 4; 6 μg/ml), and then blocked with phosphate buffered physiological saline containing 3% by weight of BSA. The chemokine provided by Dr. Melissa Swoop Wills [Vertex Pharmaceutical Co.] was used as the above C-terminal truncated secondary lymphoid tissue chemokine.

A conditioned medium of sodium chlorate-treated ACHN cells or a conditioned medium of sodium chlorate-untreated ACHN cells was added to a well of the resulting plate, followed by incubation for 1 hour. After the well was washed with the buffer A, 1 μl/ml a biotinylated anti-D antibody was added thereto, followed by incubation for 1 hour. Binding was determined by measuring absorbance at 620 nm using alkaline phosphatase-conjugated streptavidin and Blue Phos™ substrate. The results are shown in panel B of FIG. 4. In panel B of FIG. 4, a black bar shows results of the case where versican derived from untreated conditioned medium was used, and a hatched bar shows results of the case where versican derived from sodium chlorate-treated conditioned medium was used. Each bar shows mean±standard deviation of tetraplicate measurements.

As shown in panel B of FIG. 4, both of intact versican and low-sulfated versican bound to anti-versican monoclonal antibody 2B1 and CD44-Ig, and only intact versican bound with L-selectin-Ig and P-selectin-Ig, which is consistent with the results shown in FIG. 1.

In addition, as shown in panel B of FIG. 4, versican which is intact but is not low-sulfated (black bar) remarkably bound with chemokine such as secondary lymphoid tissue chemokine, γ-interferon inducible protein-10, platelet factor 4, and stromal cell-derived factor-1β. Therefore, it was suggested that sulfation in a CS/DS chain of versican is necessary for the interaction with chemokine.

Further, as shown in panel B of FIG. 4, any type of intact and low-sulfated versicans scarcely bound to recombinant truncated-type secondary lymphoid tissue chemokine lacking C-terminal 32 amino acids containing a basic amino acid cluster or, if any, slightly bound thereto. In addition, any type of intact and low-sulfated versicans did not bind to stromal cell-derived factor-1α naturally defective in C-terminal 4 amino acids of stromal cell-derived factor-1β. From these results, it was suggested that a CS/DS chain of versican interacts with each of a C-terminal region of secondary lymphoid tissue chemokine and a C-terminal region of stromal cell-derived factor-1β.

### EXAMPLE 5

Effects of a persulfated CS/DS chain on binding between versican and chemokine was examined.

A well of a 96 well flat bottom microtiter plate was coated with BSA (5 μg/ml), anti-versican monoclonal antibody 2B1 (5 μg/ml), CD44-Ig (7 μg/ml), L-selectin-Ig (3 μg/ml), P-selectin-Ig (3 μg/ml) or chemokine (1 μg/ml), and then blocked with a phosphate buffered physiological saline containing 3% by weight of BSA. As the above chemokine, secondary lymphoid tissue chemokine, γ-interferon inducible protein-10, platelet factor 4, stromal cell-derived factor-1β [manufactured by Pepro Tech] and stromal cell-derived factor-1α [manufactured by Pepro Tech] were used.

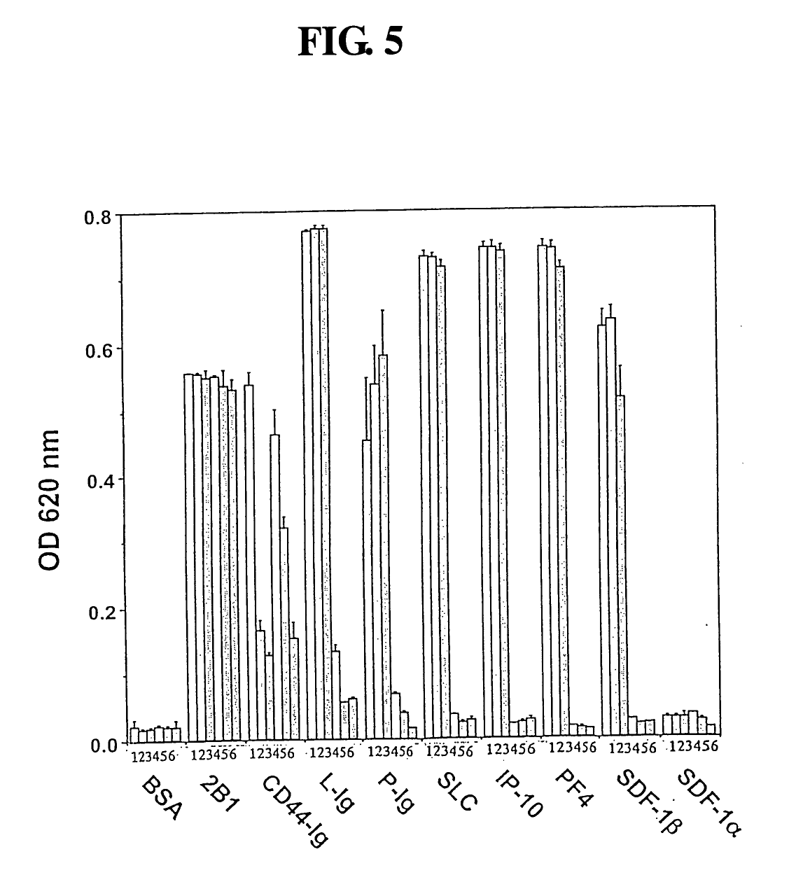

In the well on the resulting plate, biotinylated versican (0.25 μg/ml) was incubated at room temperature for 2 hours in the presence or absence of 100 μg/ml of each glycosaminoglycan (chondroitin, chondroitin sulfate A, chondroitin polysulfate, dermatan polysulfate and chondroitin sulfate E). Thereafter, the plate was washed with the buffer A, and enzyme-linked immunosorbent assay was performed in the same manner as that of the previous report [Kawashima, H. et al., J. Biol. Chem., 275, 35448-35456 (2000)]. Binding was determined by measuring absorbance at 620 nm using alkaline phosphatase-conjugated streptavidin and Blue Phos™ substrate. Results are shown in FIG. 5. In FIG. 5, expression of an abscissa axis is the same as that of FIG. 4. In each lane, bar 1 shows the results in the absence of glycosaminoglycan, bar 2 being chondroitin, bar 3 being chondroitin sulfate A, bar 4 being chondroitin polysulfate, bar 5 being dermatan polysulfate, and bar 6 being chondroitin sulfate E.

As shown in FIG. 5, each binding between biotinylated versican and each of secondary lymphoid tissue chemokine, γ-interferon inducible protein-10, platelet factor 4, stromal cell-derived factor-1β, L-selectin and P-selectin was inhibited by a persulfated CS/DS chain such as chondroitin polysulfate, dermatan polysulfate, and chondroitin sulfate E to the same degree. In addition, as shown above, although binding between biotinylated versican and CD44-Ig was inhibited by each of chondroitin, chondroitin sulfate A, dermatan polysulfate and chondroitin sulfate E, the effects of chondroitin polysulfate were not remarkable in the dose range used in the present experimental system. Binding between versican and anti-versican monoclonal antibody 2B1 was not affected by addition of any of examined glycosaminoglycans. Namely, from these results, it was suggested that sulfation of a CS/DS chain was necessary for the interaction with chemokine, and that a persulfated CS/DS chain containing GlcAβ1/IdoAα1-3GalNAc(4,6-O-disulfate) interacted with chemokine.

### EXAMPLE 6

Affinity kinetics of the interaction between various CS/DS chains and each of L-selectin, P-selectin, CD44 and chemokine were examined by surface plasmon resonance using BIAcore™ biosensor [manufactured by BIAcore AB].

All experiments were performed at 25° C. At stages of washing and dissociation, buffer C (20 mM HEPES-NaOH, 0.15M NaCl, 1 mM CaCl₂, 1 mM MgCl₂, pH6.8) was used as a running buffer.

First, about 1.8 to 2.0 kiloresonance unit (1 kiloresonance unit=1 ng/mm²) of streptavidin were covalently immobilized onto B1 sensor chip via a primary amine group using an amine coupling kit [manufactured by Amercham-Bioscience] according to the instruction provided by the manufacturer. The remaining activated groups were blocked with 150 μl of 1M ethanolamine-HCl (pH8.5). Then, each glycosaminoglycan which had been biotinylated at a reducing end was injected to a sensor tip surface by using EZ-link™ biotin-LC-hydrazide [manufactured by Pierce] so as to obtain about 150 resonance unit of an immobilization level, according to the method of Sadir et al. [J. Biol. Chem., 276, 8288-8296 (2001); all teachings of which are hereby incorporated by reference].

Binding assay was performed by injecting continuously each of various concentrations of secondary lymphoid tissue chemokine, γ-interferon inducible protein-10, stromal cell-derived factor-1β, monomeric L-selectin [manufactured by Genzyme-Techne], monomeric P-selectin [manufactured by Genzyme-Techne] and monomeric CD44, to a glycosaminoglycan-coupled sensor tip at a flow rate of 30 μl/min for 0 to 90 seconds, and then injecting a running buffer thereto. A response in resonance unit was recorded as a function of a time.

A sensor tip surface was regenerated with 300 μl of 1 M NaCl when chemokine or CD44 was used, or regenerated with 300 μl of 1 M NaCl, and additional 100 μl of 50 mM EDTA (pH8.0), when selectin was used. As a result of regeneration of a sensor tip surface, a remarkable change in a baseline was not observed.

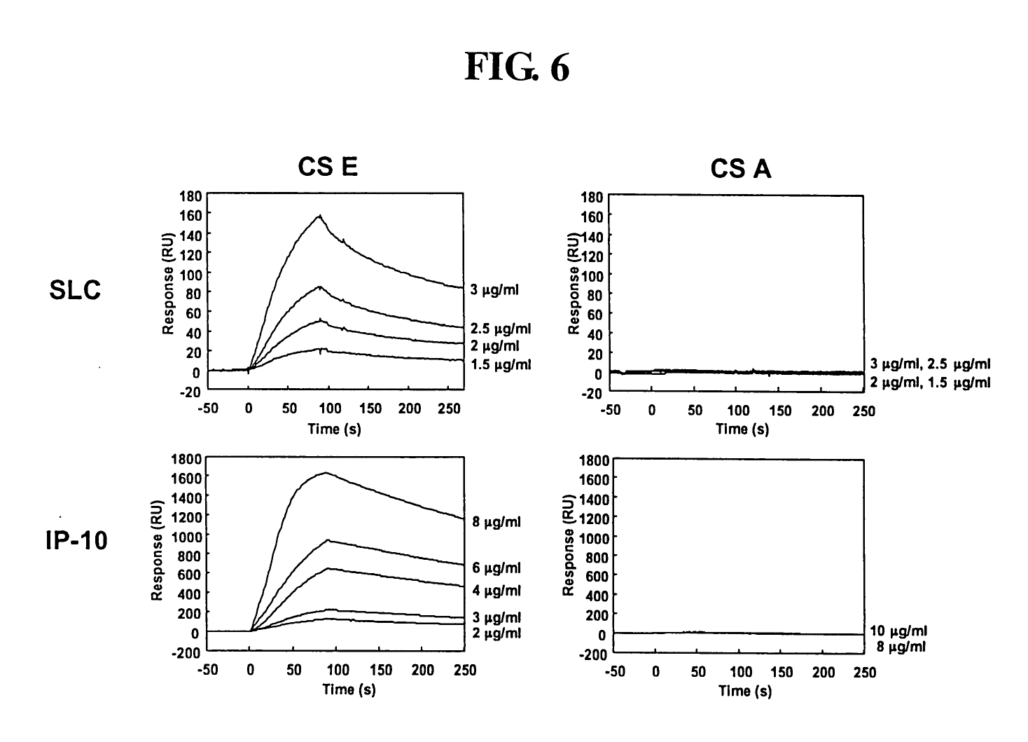

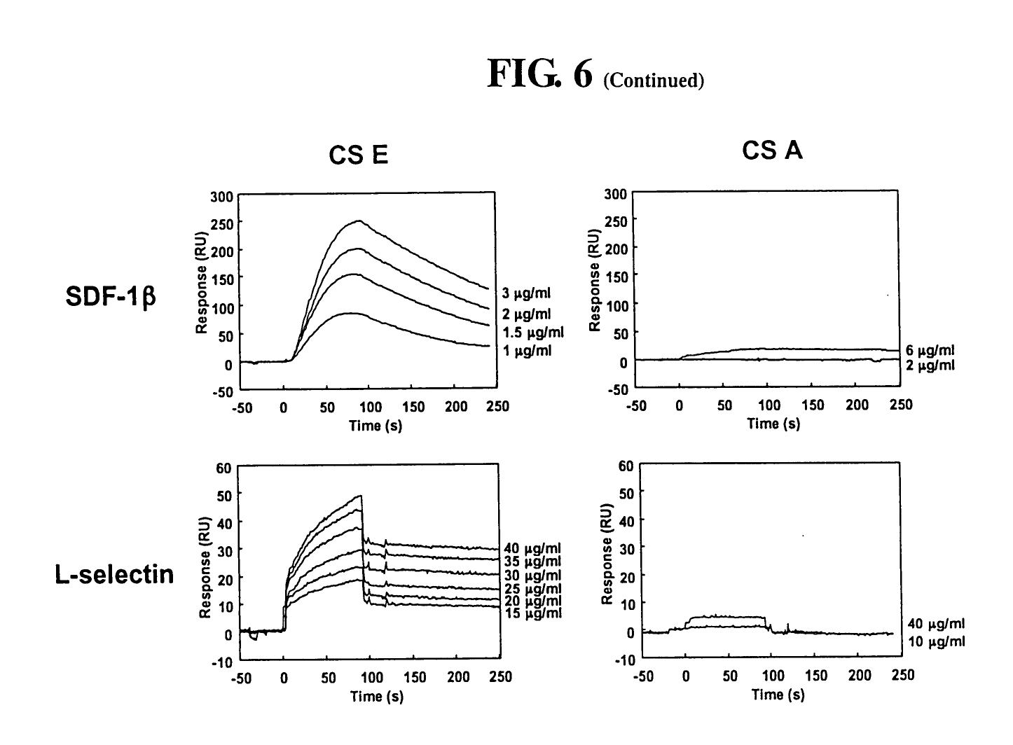

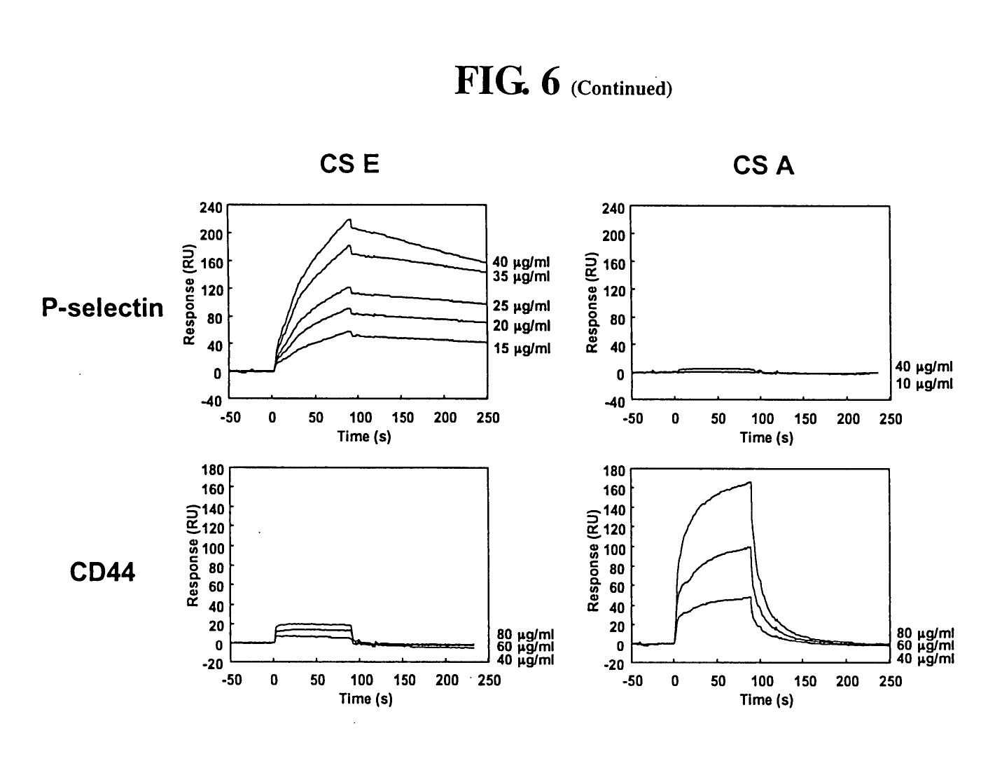

An affinity kinetic parameter was determined by BIAevaluation 3.0 software [manufactured by Pharmacia Biosensor] using a single site binding model. FIG. 6 shows a sensorgram of BIAcore recording the interaction between immobilized glycosaminoglycan, and each of chemokine, L-selectin and CD44. Table 3 shows kinetic parameters of the interaction [association rate constant (kₒₙ), dissociation rate constant (k_{off}) and equilibrium dissociation constant (k_{d})]. In FIG. 6 and Table 3, SLC denotes secondary lymphoid tissue chemokin, IP-10 denotes γ-interferon inducible protein-10, SDF-1β denotes stromal cell-derived factor-1β, CS E denotes chondroitin sulfate E, and CS A denotes chondroitin sulfate A. In addition, in Table 3, K_{d} value for the interaction between monomeric CD44 and chondroitin sulfate E is a value calculated from a binding amount at equilibrium, and K_{d} value of the interaction between CD44 and hyaluronic acid is a value calculated using CD44-Ig.

TABLE 3

| | | kₒₙ | k_{off} | K_{d} |
|---|---|---|---|---|
| Protein | GAG | (M⁻¹s⁻¹) | (s⁻¹) | (nM) |
| L-selectin | CS E | 2.07 × 10⁴ | 4.39 × 10⁻⁴ | 21.2 |
| | CPS | 7.68 × 10³ | 3.66 × 10⁻⁴ | 47.7 |
| | DPS | 1.37 × 10⁴ | 2.89 × 10⁻⁴ | 21.1 |
| P-selectin | CS E | 4.21 × 10⁴ | 1.25 × 10⁻³ | 29.7 |
| | CPS | 1.34 × 10⁴ | 7.59 × 10⁻⁴ | 56.8 |
| | DPS | 2.40 × 10⁴ | 6.64 × 10⁻⁴ | 27.7 |
| CD44 | CS E ^{a} | ND ^{b} | ND | 2.11 × 10⁵ |
| | CH | 5.03 × 10² | 6.49 × 10⁻² | 1.29 × 10⁵ |
| | CS A | 5.76 × 10² | 4.90 × 10⁻² | 8.52 × 10⁴ |
| | HA ^{c} | 2.61 × 10⁴ | 5.43 × 10⁻² | 2.08 × 10³ |
| SLC | CS E | 4.15 × 10⁴ | 3.56 × 10⁻³ | 85.8 |
| | CPS | 2.42 × 10⁴ | 2.44 × 10⁻³ | 101 |
| | DPS | 8.64 × 10³ | 2.78 × 10⁻⁴ | 32.2 |
| IP-10 | CS E | 1.62 × 10⁴ | 2.11 × 10⁻³ | 130 |
| | CPS | 3.15 × 10⁴ | 2.30 × 10⁻³ | 73.2 |
| | DPS | 2.64 × 10⁴ | 9.23 × 10⁻⁴ | 34.9 |
| SDF-1β | CS E | 1.81 × 10⁴ | 5.30 × 10⁻³ | 293 |
| | CPS | 3.52 × 10⁴ | 3.04 × 10⁻³ | 86.3 |

^{a} Kd value for the interaction between monomeric CD44 and CS E was calculated from the amount bound at equilibrium.
^{b} ND, not determined.
^{c} Kd value for the interaction between CD44 and HA was determined using CD44-Ig. Affinity kinetic parameters were determined with the BIA evaluation 3.0 software using the bivalent analyte binding model.

As shown in FIG. 6, secondary lymphoid tissue chemokine, γ-interferon inducible protein-10, stromal cell-derived factor-1β, monomeric L-selectin and monomeric P-selectin bound to chondroitin sulfate E immobilized on a sensor tip surface in a dose dependent manner. The similar affinity kinetic was observed when chondroitin polysulfate or dermatan polysulfate was used in place of chondroitin sulfate E. In contrast, selectin and chemokine did not interact with chondroitin sulfate A or chondroitin. These results were consistent with the results of the above inhibition assay shown in FIG. 2 and FIG. 5.

Further, evaluation of the affinity kinetic parameters shown in Table 3 showed that L-selectin, P-selectin and chemokine interact with a persulfated CS/DS chain containing GlcAβ1/IdoAα1-3GalNAc(4,6-O-disulfate) with high affinity (K_{d}: 21.1 to 293 nM). In contrast, it was shown that the interaction between CD44 and glycosaminoglycan is considerably different from the interaction between selectin and glycosaminoglycan, and the interaction between chemokine and glycosaminoglycan. In addition, from results of Table 3, it was shown that monomeric CD44 bound to chondroitin sulfate A or chondroitin with low affinity (K_{d}: 85.2 to 129 μM). Monomeric CD44 weakly bound to chondroitin sulfate E (K_{d}: 211 μM), and did not bind to chondroitin polysulfate or dermatan polysulfate. All of examined CS/DS chains hardly interacted with monomeric E-selectin, C-terminal truncated secondary lymphoid tissue chemokine and stromal cell-derived factor-1α, or if any, slightly interacted therewith. On the other hand, as shown in Table 3, a persulfated CS/DS chain interacted with particular chemokine with high affinity as in the case of L-selectin or P-selectin. High affinity binding between a persulfated CS/DS chain and chemokine (secondary lymphoid tissue chemokine, γ-interferon inducible protein-10, and stromal cell-derived factor-1β) suggested that these chemokines are immediately trapped by a persulfated CS/DS chain in vivo. This hypothesis is supported by kinetic analysis by surface plasmon resonance analysis showing that the formation of a persulfated CS/DS-chemokine complex is characterized by a high association rate (0.864 to 4.15×10⁴ M⁻¹s⁻¹).

Previously, it was reported that monomeric L-selectin binds to immobilized glycosylation-dependent cell adhesion molecule-1 (GlyCAM-1) with low affinity (K_{d}=108 μM) at a very high association rate (≧10⁵ M⁻¹s⁻¹) and dissociation rate (≧10⁵ s⁻¹) [Nicholson, M. W. et al., J. Biol. Chem., 273, 763-770 (1998)]. In addition, it was reported that monomeric P-selectin binds to P-selectin glycoprotein ligand-1 with relatively high affinity (K_{d}: about 300 nM) at a high association rate (4.4×10⁶ M⁻¹s⁻¹) and dissociation rate (1.4 s⁻¹) [Mehta, P. et al., J. Biol. Chem., 273, 32506-32513 (1998)]. It is presumed that these properties are important in selectin-mediated rolling adhesion dynamics mediated by rapid adhesion and deadhesion.

However, surprisingly, as shown in FIG. 6 and Table 3, the surface plasmon resonance analysis showed that binding affinity between a persulfated CS/DS chain and each of L-selectin and P-selectin is higher than binding affinity between the known ligand and each of L-selectin and P-selectin. Therefore, when an appropriate CS/DS chain is locally expressed, it is thought that high affinity binding between a persulfated CS/DS chain and each of L-selectin and P-selectin at a low dissociation rate as shown in Table 3 allows for leukocyte rolling interaction and/or static adhesion interaction at a different rolling rate.

### EXAMPLE 7

Various oligosaccharide fragments were prepared from each of chondroitin sulfate A, chondroitin sulfate C and chondroitin E by digestion with ovine testis hyaluronidase, to determine a structural unit which directly interacts with each of L-selectin, P-selectin, CD44 and chemokine.

(1) Preparation of Streptavidin-Conjugated Alkaline Phosphatase-Conjugated Biotinylated Oligosaccharide Fragment Derived from any of Chondroitin Sulfate A, Chondroitin Sulfate C and Chondroitin Sulfate E

Squid cartilage-derived chondroitin sulfate E (1 mg) was suspended in a solution (composition: 50 mM sodium acetate, 133 mM NaCl and 0.04% gelatin, pH 5.0) containing 0.6 mg of ovine testis hyaluronidase [1,800 units; manufactured by Sigma]. The resulting reaction solution was incubated at 37° C. for a total of 68.5 hours to obtain the digestion product containing a chondroitin sulfate E-derived oligosaccharide fragment. After 24 hours and 45.5 hours from initiation of incubation, additional 2 mg (6,000 units) of the enzyme was added to the reaction solution.

In addition, each of whale cartilage-derived chondroitin sulfate A and shark cartilage-derived chondroitin sulfate C was suspended in a solution (composition: 50 mM sodium acetate, pH 5.0) containing 0.6 mg of ovine testis hyaluronidase [1,800 units; manufactured by Sigma]. Each of the resulting reaction solutions was incubated at 37° C. for 24 hours to obtain each of the digestion products containing a chondroitin sulfate A-derived oligosaccharide fragment and the digestion product containing a chondroitin sulfate C-derived oligosaccharide fragment.

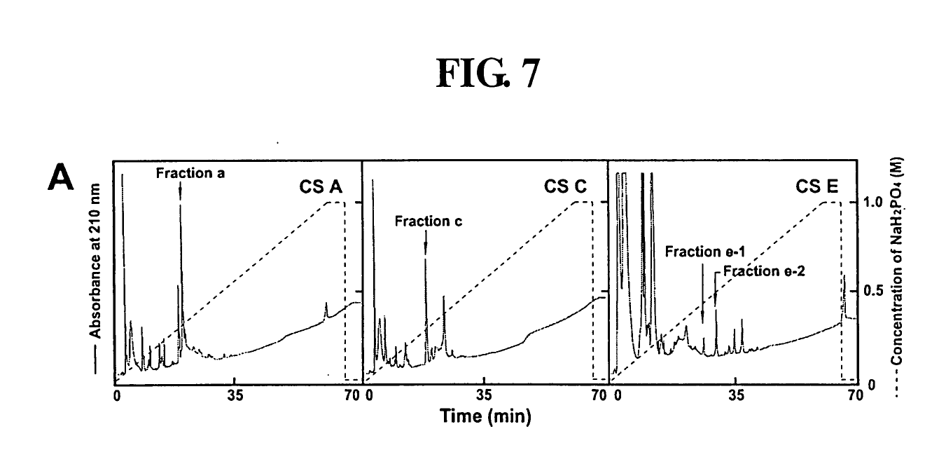

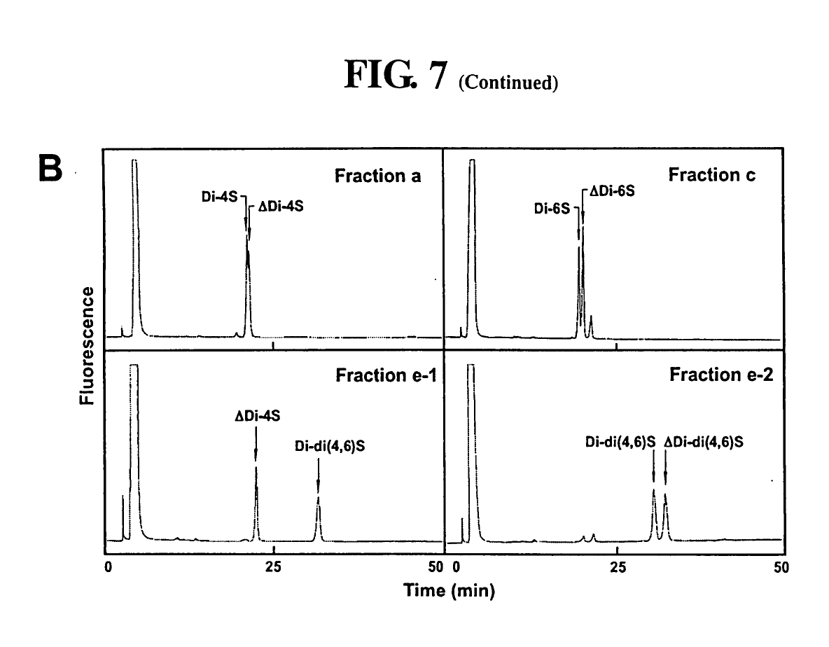

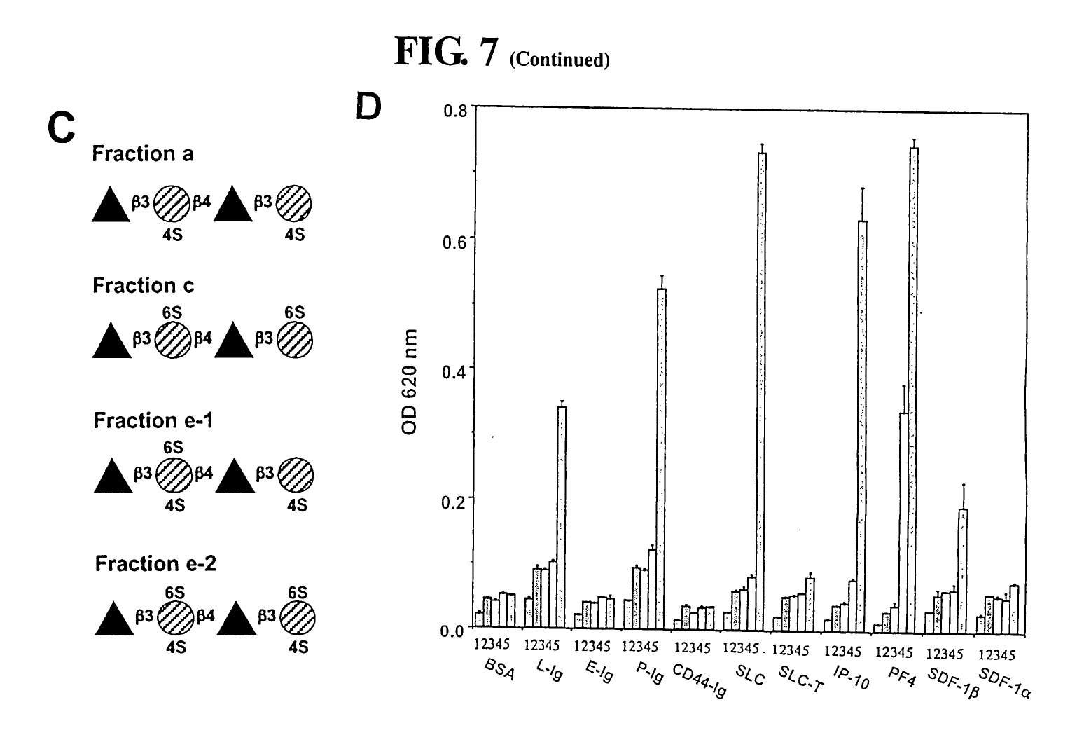

Each of the resulting digest products was fractionated by HPLC with an amine-coupled silica PA-03 column using a linear gradient of 16 mM to 1M NaH₂PO₄. The resulting fractions were respectively subjected to Sephadex G-25 column [1×30 cm; manufactured by Amersham-Biosciences] equilibrated with distilled water. Elution was monitored by absorbance at 210 nm. As a result, the fraction a, the fraction c, the fraction e-1 and the fraction e-2 shown in panel A of FIG. 7 were obtained.

(2) Analysis of Hydrocarbon Structure of Oligosaccharide Fragment

Each of the fragment a, the fragment c, the fragment e-1 and the fragment e-2 was digested with chondroitinase ACII (0.3 unit/ml) at 37° C. for 1 hour, and thereafter, the resulting digestion product was derivatized with 2-AB. Then, each of the resulting products was analyzed by HPLC with amine-coupled silica PA-03 column with a linear gradient elution of 16 to 606 mM NaH₂PO₄ for 45 minutes.

As shown in panel B of FIG. 7, regarding a chodroitinase ACII-treated fraction e-1, Di-di(4,6)S and ΔDi-4S were detected at a molar ratio of about 1:1. These peaks were shifted to Di-4S and ΔDi-4S positions, respectively, after treatment with a mixture of chondroitinase ACII and chondro-6-sulfatase, and further shifted to Di-0S and ΔDi-0S positions, respectively, after treatment with a mixture of chondroitinase ACII, chondro-6-sulfatase and chondro-4-sulfatase. Therefore, these results suggested that the fraction e-1 corresponds to the structure of GlcAβ1-3GalNAc(4,6-O-disulfate)β1-4GlcAβ1-3GalNAc(4-O-sulfate). Such structure was also supported by mass spectrum.

Similarly, it was determined that the structure of the fraction a corresponds to GlcAβ1-3GalNAc(4-O-sulfate)β1-4GlcAβ1-3GalNAc(4-O-sulfate), that the structure of the fraction c corresponds to GlcAβ1-3GalNAc(6-O-sulfate)β1-4GlcAβ1-3GalNAc(6-O-sulfate), and that the structure of the fraction e-2 corresponds to GlcAβ1-3GalNAc(4,6-O-disulfate)β1-4GlcAβ1-3GalNAc(4,6-O-disulfate).

The above results are shown as a schematic view of an oligosaccharide structure in panel C of FIG. 7. In panel C of FIG. 7, a solid triangle denotes GlcA, a hatched circle being GalNAc, 4S being 4-O-sulfation, 6S being 6-O-sulfation, β3 being β1-3 linkage, and β4 being β1-4 linkage.

(3) Analysis of the Interaction Between Oligosaccharide Fragment and any of L-Selectin, P-Selectin, CD44 and Chemokine

The interaction of an oligosaccharide contained in any of the fraction a, the fraction c, the fraction e-1 and the fraction e-2, with any of L-selectin, P-selectin, CD44 and chemokine was examined.

First, each of oligosaccharides was biotinylated with biotin-LC-hydrazide at a reducing end. To each of the fraction a, the fraction c, the fraction e-1 and the fraction e-2 obtained in the above item (2), 125 mM EZ-link™ biotin-LC-hydrazide and 1M NaCNBH₃ in dimethyl sulfoxide/acetic acid (7:3) were added. The resulting reaction mixture was incubated at 65° C. for 3 hours, and then incubated at 37° C. for 12.5 to 18.5 hours to biotinylate an oligosaccharide. Each of the resulting reaction products was subjected to Sephadex G-25 column as described above, to remove unreacted biotin-LC-hydrazide, thereby collecting the fraction containing a biotinylated oligosaccharide. Then, the resulting fraction was evaporated to dryness.

A part of the resulting biotinylated oligosaccharide fraction was digested with chondrotinase ACII. Then, the resulting digestion product was derivatized with 2-AB. As a result of HPLC analysis of the resulting product, a non-reducing end-derived disaccharide was detected rather than a reducing end-derived unsaturated disaccharide. Therefore, it was made clear that about 100% oligosaccharide was biotinylated.

Then, a part of a fraction containing 16 pmol of each biotinylated oligosaccharide was dissolved in 0.5 ml of the buffer A, and thereafter, each of the resulting solutions and 1 μl (2 pmol) of streptavidin-conjugated alkaline phosphatase [manufactured by Promega] were incubated at 4° C. overnight.

Each of the resulting products was 3-fold diluted with the buffer A respectively. Thereafter, the resulting solution was subjected to a well of a 96 well flat bottom microtiter plate (Coster EIA/RIA plate number 3690) coated with BSA (10 μg/ml), L-selectin-Ig (5 μg/ml), E-selectin-Ig (5 μg/ml), P-selectin-Ig (5 μg/ml), CD44-Ig (5 μg/ml), secondary lymphoid tissue chemokine (5 μg/ml), C-terminal truncated secondary lymphoid tissue chemokine (5 μg/ml), γ-interferon inducible protein-10 (10 μg/ml), platelet factor 4 (2.5 μg/ml), stromal cell-derived factor/1β (5 μg/ml) or stromal cell-derived factor-1α (5 μg/ml). Binding was determined by measuring absorbance at 620 nm using Blue Phos™ substrate. Results are shown in panel D of FIG. 7. In panel D of FIG. 7, expression of an abscissa axis is the same as that of FIG. 4. In addition, in each of lane, bar 1 denotes the results of the case where streptavidin-conjugated alkaline phosphatase is used, bar 2 being the results of the case where biotinylated fraction a-conjugated streptavidin-conjugated alkaline phosphatase is used, bar 3 being the results of the case where biotinylated fraction c-conjugated streptavidin-conjugated alkaline phosphatase is used, bar 4 being the results of the case where biotinylated fraction e-1-conjugated streptavidin-conjugated alkaline phophatase is used, and bar 5 being the results of the case where biotinylated fraction e-2-conjugated streptavidin-conjugated alkaline phosphatase is used.

As shown in panel D of FIG. 7, interestingly, only the e-2 fraction was bound to L-selectin-Ig, P-selectin-Ig, secondary lymphoid tissue chemokine, γ-interferon inducible protein-10 and stromal cell-derived factor-1β, but the others did not bind thereto. The e-1 fraction moderately bound to platelet factor 4, but did not bind to the other chemokines, L-selectin or P-selectin. These results showed that a repeating GlcAβ1-3GalNAc(4-6-O-disulfate) unit is specifically recognized by L-selectin, P-selectin and many chemokines examined, and that GlcAβ1-3GalNAc(4,6-O-disulfate) alone is probably sufficient for the interaction with platelet factor 4.

Since each of L-selectin, P-selectin and chemokine is preferentially bound to a tetrasaccharide composed of a repeating GlcAβ1-3GalNAc(4,6-O-disulfate) unit as shown in FIG. 7, and since CD44 interacts preferentially with an unsulfated chondroitin sulfate chain or a low-sulfated chondroitin sulfate chain as shown in Table 3, it is considered that, when a GlcAβ1-3GalNAc(4,6-O-disulfate) unit is present as a cluster in glycosamanoglycan, probably, these units interact with each of L-selectin, P-selectin and chemokine. In addition, it is considered that a different structure containing GlcAβ1-3GalNAc(4-O-sulfate) or GlcAβ1-3GalNAc(6-O-sulfate) may interact with CD44.

### EXAMPLE 8

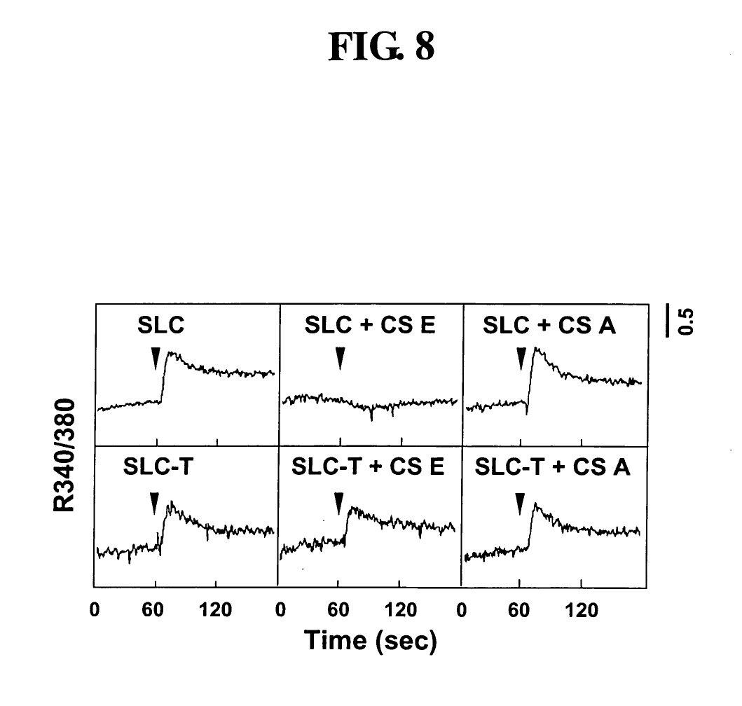

Whether a persulfated CS/DS chain also inhibits chemokine activity or not was examined. L1.2/CCR7 cells (1×10⁶ cells/ml) were loaded with Fura-2, and then stimulated with secondary lymphoid tissue chemokine (“SLC” in FIG. 8) or C-terminal truncated secondary lymphoid tissue chemokine (“SLC-T” in FIG. 8), in the presence or absence of glycosaminoglycan (100 μg/ml). The intracellular calcium concentration was monitored by measuring a fluorescent rate in the same manner as described in the previous report [Hirose, J. et al., J. Biol. Chem., 276, 5228-5234 (2001)]. Chondroitin sulfate E (“CS E” in FIG. 8) and chondoroitin sulfate A (“CS A” in FIG. 8) were used as the glycosaminoglycan. The results are shown in FIG. 8. In FIG. 8, an arrowhead indicates a time point at which stimulation was given.

As shown in FIG. 8, secondary lymphoid tissue chemokine alone, or secondary lymphoid tissue chemokine which had been pre-incubated with chondroitin sulfate A remarkably induced Ca²⁺ mobilization in L1.2 cells in which a receptor of the secondary lymphoid tissue chemokine, CCR7 had been incorporated by transfection, but secondary lymphoid tissue chmokine which had been pre-incubated with chondoroitin sulfate E did not induce Ca²⁺ mobilization. Similarly, secondary lymphoid tissue chemokine which had been pre-incubated with chondoroitin polysulfate or dermatan polysulfate did not induce Ca²⁺ mobilization. On the other hand, Ca²⁺ mobilization induced by C-terminal truncated secondary lymphoid tissue chemokine was not affetced by any of these persulfated CS/DS chains. These results suggested that a persulfated CS/DS chain inhibits physiological activity of secondary lymphoid tissue chemokine by the interaction with a C-terminal region of secondary lymphoid tissue chemokine.

In addition, as shown in FIG. 8, since these glycosaminoglycans inhibit chemokine activity, it is thought that persulfated CS/DS-conjugated chemokine may not function as an agonist for a chemokine receptor, but rather a persulfated CS/DS-chemokine complex may function as a reserver of chemokine in vivo. A low dissociation rate (2.78×10⁻⁴ to 5.30×10⁻³ s⁻¹) observed in the interaction between chemokine and a persulfated CS/DS chain supports this idea.

Sequence Listing Free Text

SEQ ID No.: 1 shows a sequence of a primer for amplifying CD44 gene.

SEQ ID No.: 2 shows a sequence of a primer for amplifying CD44 gene.

### INDUSTRIAL APPLICABILITY

The saccharide compound of the present invention can be easily prepared upon preparation thereof. In addition, according to the saccharide compound of the present invention, there can be achieved the regulation of the binding of any of L-selectin, P-selectin and chemokine to the ligand thereof, the regulation of biological events mediated by any of L-selectin, P-selectin and chemokine, improvement in symptom of a disease of which sideration is associated with the biological events, and provision of a lead compound for a therapeutic or prophylactic agent for the disease. Further, the therapeutic or prophylactic agent of the present invention is useful for treating or preventing a disease of which sideration is associated with biological events mediated by any of L-selectin, P-selectin and chemokine, such as inflammatory disease, allergic disease, cancer metastasis, myocardial dysfunction, and multiple organ failure.

1. A saccharide compound represented by the general formula (I): 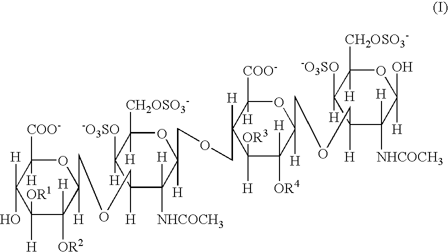
wherein R¹, R², R³ and R⁴ each independently represent a hydrogen atom or a sulfonic group, or the general formula (II): 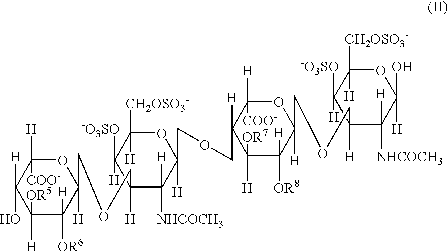
wherein R⁵, R⁶, R⁷ and R⁸ each independently represent a hydrogen atom or a sulfonic group.

2. A saccharide compound represented by the general formula (III): 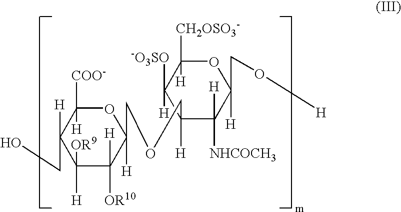
wherein R⁹ and R¹⁰ each independently represent a hydrogen atom or a sulfonic group, and m is 3 or 4, or the general formula (IV): 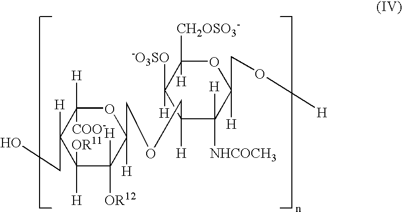
wherein R¹¹ and R¹² each independently represent a hydrogen atom or a sulfonic group, and n is 3 or 4.

3. A pharmaceutical composition comprising the saccharide compound of claim 1 or 2 as an active ingredient.

4. An agent for treating or preventing a disease of which sideration is associated with biological events mediated by any of L-selectin, P-selectin and chemokine, wherein said agent comprises the saccharide compound of claim 1 or 2 as an active ingredient.
